(19) United States
(12) Patent Application Publication
(10) Pub. No.: US 2024/0082242 A1
(43) Pub. Date: Mar. 14, 2024

(54) COMBINATION THERAPIES

(71) Applicant: IMBRIA PHARMACEUTICALS, INC., Boston, MA (US)

(72) Inventors: Paul Chamberlin, Brookline, MA (US); Jaikrishna Patel, Cary, NC (US)

(21) Appl. No.: 18/269,820

(22) PCT Filed: Jan. 5, 2022
(86) PCT No.: PCT/US2022/011336

§ 371 Date: Jun. 27, 2023

Related U.S. Application Data
(60) Provisional application No. 63/147,801, filed on Feb. 10, 2021.
(60) Provisional application No. 63/134,243, filed on Jan. 6, 2021.

(51) Int. Cl. A61K 31/496; A61K 31/495; A61P 9/04; A61P 9/10

(52) U.S. Cl. CPC A61K 31/496; A61K 31/495; A61P 9/04; A61P 9/10

(57) ABSTRACT

The invention provides combination therapies that include CV-8972 and CV-8814. The combination therapies improve cardiac efficiency and thus are useful for treating cardiovascular conditions in a subject. The invention also provides methods of treating conditions using both CV-8972 and CV-8814 and pharmaceutical compositions that contain both CV-8972 and CV-8814.

### FIELD OF THE INVENTION

The invention relates to combination therapies, methods of treating conditions, and pharmaceutical compositions, all of which include both CV-8972 and CV-8814.

### BACKGROUND

Cardiovascular disease is the leading cause of death worldwide, accounting for an estimated 17.9 million deaths across the globe in 2019 (WHO). In many forms of heart disease, decreased cardiac efficiency stems from changes in mitochondrial energy metabolism. Mitochondria are sub-cellular compartments in which metabolites derived from glucose and fatty acids are oxidized to produce high-energy molecules. Increasing fatty acid oxidation in the heart decreases glucose oxidation, and vice versa. Glucose oxidation is a more oxygen-efficient source of energy, but in certain types of heart disease, such as heart failure, ischemic heart disease, and diabetic cardiomyopathies, there is an excessive reliance on fatty acid oxidation which predominates in cardiac mitochondria and/or uncoupling of glycolysis from glucose oxidation. As a result, the efficiency of energy generation and production of ATP is reduced, with the corollary that the pumping capacity of the heart is reduced.

The compound 2-[4-[(2,3,4-trimethoxyphenyl)methyl]piperazin-1-yl]ethyl pyridine-3-carboxylate, referred to herein as CV-8972, was recently identified as therapeutic candidate to improve cardiac efficiency in patients with a variety of cardiovascular conditions. CV-8972 is a prodrug that is broken down in the body into multiple metabolic products that exert distinct and synergistic effects to promote energy production by cardiac mitochondria. The metabolic products of CV-8972 include niacin, 2-[4-[(2,3,4-trimethoxyphenyl)methyl]piperazin-1-yl]ethanol, referred to herein as CV-8814, and trimetazidine. The latter two products are generated sequentially from CV-8972 catabolism. CV-8972 is initially hydrolyzed to yield CV-8814 and niacin, and the ethylene glycol moiety of CV-8814 is subsequently removed to produce trimetazidine. Both CV-8814 and trimetazidine inhibit oxidation of fatty acids and thus force the heart to derive energy from oxidation of glucose instead. Niacin is converted in the body to nicotinamide adenine dinucleotide (NAD⁺), which facilitates the transfer of electrons in the mitochondria to enable cells to derive energy from molecular oxygen. Consequently, providing the heart with niacin maximizes the heart's ability to produce energy from its oxygen supply. Thus, metabolism of CV-8972 is bifurcated to produce molecules that stimulate glucose oxidation and a molecule that serves as a NAD⁺ precursor, and the two classes of CV-8972 metabolic products act in a complementary manner to stimulate cardiac efficiency.

### SUMMARY

The invention provides therapeutic combinations of CV-8972 and CV-8814 that allow delivery of the two classes of CV-8972 metabolic products in optimized ratios. The invention recognizes that each molecule of CV-8972 administered to a patient yields CV-8814 and niacin in equimolar amounts and that optimal therapeutic intervention often requires CV-8814:niacin ratios of greater than unity. First, CV-8814 is relatively well-tolerated by the body due to its pharmacological properties. Second, the conversion of CV-8814 to trimetazidine in vivo is gradual, so the peak systemic level of trimetazidine following a dose of CV-8814 is lower and later than the peak following a comparable dose of unadulterated trimetazidine. Compared to CV-8814, however, niacin is not as well-tolerated on a molar equivalence basis. Administration of high doses of niacin produces flushing and other side effects, and it is the niacin-mediated effects that limit the dosage at which CV-8972 can be administered.

The invention solves this problem through the use of combination therapies that include administration of CV-8972 and CV-8814 as distinct compounds. The combination therapies are based on the recognition that the metabolic products that result from breakdown of CV-8972 perform different functions and that uncoupling those functions allows remediation of cardiovascular conditions with greater precision. Whereas metabolism of CV-8972 yields both niacin and other molecules, viz., CV-8814 and trimetazidine, that promote glucose oxidation, metabolism of CV-8814 only produces the latter. By providing CV-8972 and CV-8814 as separate therapeutic agents, dosing of the NAD⁺ precursor niacin and dosing of the products that promote glucose oxidation can be adjusted independently. Consequently, the combination therapies permit each of the active metabolic products of these drugs to be delivered at optimal therapeutic levels. The therapies and methods of the invention are useful for treating a wide range of cardiovascular conditions.

In one aspect, the invention provides combination therapies that include a compound represented by formula (X):

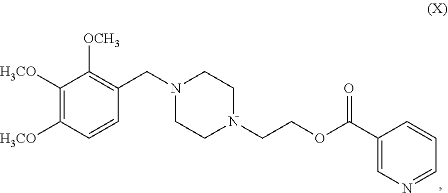 [SMILES: COc1ccc(CN2CCN(CCOC(=O)c3cccnc3)CC2)c(OC)c1OC]

or a pharmaceutically acceptable salt thereof, and a compound represented by formula (IX):

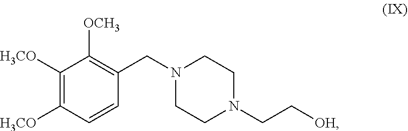 [SMILES: COc1ccc(CN2CCN(CCO)CC2)c(OC)c1OC]

or a pharmaceutically acceptable salt thereof.

The combination therapy may include compounds of formulas (X) and (IX) in a defined mass ratio. The mass ratio of the compound of formula (X) to the compound of formula (IX) may be about 10:1, about 5:1, about 2:1, about 1:1, about 1:2, about 1:5, about 1:10, about 1:20, about 1:50, or about 1:100. The mass ratio of the compound of formula (X) to the compound of formula (IX) may be from about 10:1 to about 5:1, from about 10:1 to about 2:1, from about 10:1 to about 1:1, from about 10:1 to about 1:2, from about 10:1 to about 1:5, from about 10:1 to about 1:10, from about 10:1 to about 1:20, from about 10:1 to about 1:50, from about 10:1 to about 1:100, from about 5:1 to about 2:1, from about 5:1 to about 1:1, from about 5:1 to about 1:2, from about 5:1 to about 1:5, from about 5:1 to about 1:10, from about 5:1 to about 1:20, from about 5:1 to about 1:50, from about 5:1 to about 1:100, from about 2:1 to about 1:1, from about 2:1 to about 1:2, from about 2:1 to about 1:5, from about 2:1 to about 1:10, from about 2:1 to about 1:20, from about 2:1 to about 1:50, from about 2:1 to about 1:100, from about 1:1 to about 1:2, from about 1:1 to about 1:5, from about 1:1 to about 1:10, from about 1:1 to about 1:20, from about 1:1 to about 1:50, from about 1:1 to about 1:100, from about 1:2 to about 1:5, from about 1:2 to about 1:10, from about 1:2 to about 1:20, from about 1:2 to about 1:50, from about 1:2 to about 1:100, from about 1:5 to about 1:10, from about 1:5 to about 1:20, from about 1:5 to about 1:50, from about 1:5 to about 1:100, from about 1:10 to about 1:20, from about 1:10 to about 1:50, from about 1:10 to about 1:100, from about 1:20 to about 1:50, from about 1:20 to about 1:100, or from about 1:50 to about 1:100.

The combination therapy may include one or both of the compounds of formulas (X) and (IX) in defined daily dosages. The daily dosages of the compounds of formulas (X) and (IX) may each independently be about 10 mg, about 20 mg, about 50 mg, about 100 mg, about 200 mg, about 500 mg, about 1000 mg, about 2000 mg, or about 5000 mg. The daily dosages of the compounds of formulas (X) and (IX) may each independently be from about 10 mg to about 20 mg, from about 10 mg to about 50 mg, from about 10 mg to about 100 mg, from about 10 mg to about 200 mg, from about 10 mg to about 500 mg, from about 10 mg to about 1000 mg, from about 10 mg to about 2000 mg, from about 10 mg to about 5000 mg, from about 20 mg to about 50 mg, from about 20 mg to about 100 mg, from about 20 mg to about 200 mg, from about 20 mg to about 500 mg, from about 20 mg to about 1000 mg, from about 20 mg to about 2000 mg, from about 20 mg to about 5000 mg, from about 50 mg to about 100 mg, from about 50 mg to about 200 mg, from about 50 mg to about 500 mg, from about 50 mg to about 1000 mg, from about 50 mg to about 2000 mg, from about 50 mg to about 5000 mg, from about 100 mg to about 200 mg, from about 100 mg to about 500 mg, from about 100 mg to about 1000 mg, from about 100 mg to about 2000 mg, from about 100 mg to about 5000 mg, from about 200 mg to about 500 mg, from about 200 mg to about 1000 mg, from about 200 mg to about 2000 mg, from about 200 mg to about 5000 mg, from about 500 mg to about 1000 mg, from about 500 mg to about 2000 mg, from about 500 mg to about 5000 mg, from about 1000 mg to about 2000 mg, from about 1000 mg to about 5000 mg, or from about 2000 mg to about 5000 mg.

The compounds of formulas (X) and (IX) may be contained in separate formulations. The compounds of formulas (X) and (IX) may be contained in a single formulation.

The combination therapy may be useful for treating any disease, disorder, or condition for which increased cardiac efficiency provides a therapeutic benefit. The disease, disorder, or condition may be a cardiovascular condition. The disease, disorder, or condition may be acute coronary syndrome, acute heart failure, advanced heart failure, aneurysm, angina, anthracycline-induced cardiotoxicity, atherosclerosis, cardiac allograft vasculopathy, cardiac steatosis, cardiac transplant vasculopathy, cardiomyopathy, cerebral vascular disease, chronic coronary syndrome, chronic heart failure, congenital heart disease, contrast nephropathy, coronary artery disease (CAD), coronary heart disease, diabetic cardiomyopathy, dilated cardiomyopathy (DCM, including idiopathic, heart attack, heart disease, heart failure with mildly reduced ejection fraction (HFmrEF), heart failure with preserved ejection fraction (HFpEF), heart failure with reduced ejection fraction (HFrEF), heart failure, hibernating myocardium, high blood pressure (hypertension), hypertrophic cardiomyopathy (HCM, including non-obstructive or obstructive), intermittent claudication, ischemia with non-obstructive coronary arteries (INOCA), ischemia-reperfusion injury, ischemic cardiomyopathy, ischemic heart disease, microvascular angina, myocardial dysfunction induced by anti-cancer drugs, myocardial infarction with non-obstructive coronary arteries (MINOCA), myocarditis, non-familial and familial/genetic), non-ischemic cardiomyopathy, pericardial disease, peripartum cardiomyopathy, peripheral arterial disease, peripheral vascular disease, pulmonary arterial hypertension, pulmonary hypertension, refractory angina, restrictive cardiomyopathy, rheumatic heart disease, right heart failure, right ventricular failure, stable angina, stroke, stunned myocardium, tachycardiomyopathy, Takotsubo cardiomyopathy, transient ischemic attack, unstable angina, or valvular heart disease.

In another aspect, the invention provides methods of treating a disease, disorder, or condition in a subject by providing to the subject having the disease, disorder, or condition a compound represented by formula (X):

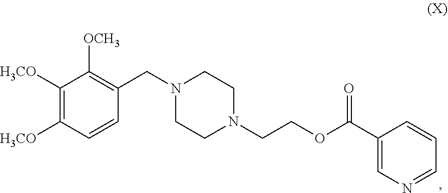 [SMILES: COc1ccc(CN2CCN(CCOC(=O)c3cccnc3)CC2)c(OC)c1OC]

or a pharmaceutically acceptable salt thereof, and a compound represented by formula (IX):

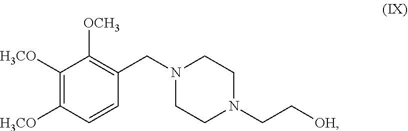 [SMILES: COc1ccc(CN2CCN(CCO)CC2)c(OC)c1OC]

or a pharmaceutically acceptable salt thereof.

The compounds of formulas (X) and (IX) may be provided in a defined mass ratio, such as any of those described above.

Each of the compounds of formulas (X) and (IX) may be independently provided at a defined daily dosage, such as any of those described above.

The compounds of formulas (X) and (IX) may be provided in separate formulations. The compounds of formulas (X) and (IX) may be provided in a single formulation.

The disease, disorder, or condition may be any disease, disorder, or condition for which increased cardiac efficiency provides a therapeutic benefit, such as any of those described above.

In another aspect, the invention provides pharmaceutical compositions that include a compound represented by formula (X):

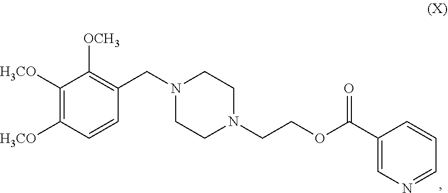 [SMILES: COc1ccc(CN2CCN(CCOC(=O)c3cccnc3)CC2)c(OC)c1OC]

or a pharmaceutically acceptable salt thereof, and a compound represented by formula (IX):

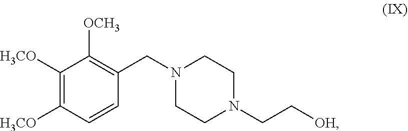 [SMILES: COc1ccc(CN2CCN(CCO)CC2)c(OC)c1OC]

or a pharmaceutically acceptable salt thereof.

The pharmaceutical composition may include the compounds of formulas (X) and (IX) in a defined mass ratio, such as any of those described above.

The pharmaceutical composition may include one or both of the compounds of formulas (X) and (IX) in defined daily dosages, such as any of those described above.

The pharmaceutical composition may be formulated for a particular route or mode of delivery. The pharmaceutical composition may be formulated for administration buccally, by injection, dermally, enterally, intraarterially, intravenously, nasally, orally, parenterally, pulmonarily, rectally, subcutaneously, topically, transdermally, or with or on an implantable medical device (e.g., stent or drug-eluting stent or balloon equivalents).

The pharmaceutical composition may be a modified release formulation.

The pharmaceutical composition may be suitable for treating a particular disease, disorder, or condition, such as any of those described above.

### BRIEF DESCRIPTION OF THE DRAWINGS

FIG. 1 is a schematic of the positron emission tomography (PET) imaging study design used to monitor the uptake of ¹⁸F-fluorodeoxyglucose (¹⁸F-FDG) and ¹⁸F-14(R,S)-(18)F-fluoro-6-thia-heptadecanoic acid (¹⁸F-FTHA) into heart tissue of rats in response to treatment with CV-8972.

FIG. 2 is a graph showing the mean standardized uptake value (SUV) of FDG in the heart at various time points following administration of either saline or CV-8972 to rats.

FIG. 3 is a graph showing the mean SUV of FDG in skeletal muscle at various time points following administration of saline or CV-8972 to rats.

FIG. 4 is a graph showing the mean SUV of FDG in the blood at various time points following administration of either saline or CV-8972 to rats.

FIG. 5 is a graph showing the mean SUV of FTHA in the heart at various time points following administration of either saline or CV-8972 to rats.

FIG. 6 is a graph showing the mean SUV of FTHA in skeletal muscle at various time points following administration of either saline or CV-8972 to rats.

FIG. 7 is a graph showing the mean SUV of FTHA in the blood at various time points following administration of either saline or CV-8972 to rats.

FIG. 8 shows PET/CT images FDG and FTHA following administration of either saline or CV-8972 to rats.

FIG. 9 is a graph showing the mean SUV of FDG in the heart, skeletal muscle, and blood in the last 30 minutes of the dynamic acquisition following administration of either saline or CV-8972 to rats.

FIG. 10 is a graph showing the mean SUV of FTHA in the heart, skeletal muscle, and blood in the last 30 minutes of the dynamic acquisition following administration of either saline or CV-8972 to rats.

FIG. 11 is a graph of FDG uptake in the myocardium following administration of either saline or CV-8972 to rats.

FIG. 12 is a graph of flow of activity of FDG in the myocardium following administration of either saline or CV-8972 to rats.

FIG. 13 is a graph of FDG uptake in skeletal muscle following administration of either saline or CV-8972 to rats.

FIG. 14 is a graph of flow of activity of FDG in skeletal muscle following administration of either saline or CV-8972 to rats.

FIG. 15 is a graph of FTHA uptake in the myocardium following administration of either saline or CV-8972 to rats.

FIG. 16 is a graph of pharmacokinetic parameter V₁ of FTHA uptake in the myocardium following administration of either saline or CV-8972 to rats.

FIG. 17 is a graph of pharmacokinetic parameter V₂ of FTHA uptake in the myocardium following administration of either saline or CV-8972 to rats.

FIG. 18 is a graph of flow of activity of FTHA in the myocardium following administration of either saline or CV-8972 to rats.

FIG. 19 is a graph of FTHA uptake in skeletal muscle following administration of either saline or CV-8972 to rats.

FIG. 20 is a graph of pharmacokinetic parameter V₁ of FTHA uptake in skeletal muscle following administration of either saline or CV-8972 to rats.

FIG. 21 is a graph of pharmacokinetic parameter V₂ of FTHA uptake in skeletal muscle following administration of either saline or CV-8972 to rats.

FIG. 22 is a graph of flow of activity of FTHA in skeletal muscle following administration of either saline or CV-8972 to rats.

FIG. 23 is a graph of gamma radioactivity from FDG in the heart, skeletal muscle, and blood following administration of either saline or CV-8972 to rats.

FIG. 24 is a graph of gamma radioactivity from FTHA in the heart, skeletal muscle, and blood following administration of either saline or CV-8972 to rats.

FIG. 25 is a schematic of the Langendorff Ischemia-Reperfusion protocol used to test the ability of various compounds to protect the heart from ischemic injury.

FIG. 26 is a graph of coronary flow during ischemia-reperfusion injury in explanted mouse hearts treated with either saline or 20 μM CV-8814.

FIG. 27 is a graph of infarct size following ischemia-reperfusion injury in explanted mouse hearts treated with either saline or 20 μM CV-8814.

FIG. 28 is a graph of infarct size following ischemia-reperfusion injury in explanted mouse hearts treated with either saline or 20 μM trimetazidine.

FIG. 29 is a graph of infarct size following ischemia-reperfusion injury in explanted mouse hearts treated with saline, 20 μM trimetazidine, 20 μM each of trimetazidine+nicotinamide+succinate, or 20 μM each of trimetazidine+nicotinic acid+succinate.

FIG. 30 is a schematic of the transverse aortic constriction (TAC) protocol used to test the ability of various compounds to protect the heart against heart failure.

FIG. 31 shows images of hearts from mice following TAC-induced heart failure.

FIG. 32 is a graph of heart weight to body weight following TAC-induced heart failure in mice treated with saline, trimetazidine, nicotinic acid, CV-8814, or CV-8972.

FIG. 33 is a graph of cardiac ejection fraction at various time points following TAC-induced heart failure in mice treated with either saline or CV-8972.

FIG. 34 is a graph of cardiac ejection fraction at various time points following TAC-induced heart failure in mice treated with either saline or CV-8814.

FIG. 35 is a graph of cardiac ejection fraction at various time points following TAC-induced heart failure in mice treated with either saline or trimetazidine.

FIG. 36 shows microscopic images of heart tissue from mice following TAC-induced heart failure.

FIG. 37 is graph of cardiac fibrosis following TAC-induced heart failure in mice treated with saline, trimetazidine, nicotinic acid, CV-8814, or CV-8972.

FIG. 38 is a schematic of a two-compartment model.

FIG. 39 is a schematic of a three-compartment model.

### DETAILED DESCRIPTION

In many forms of heart disease, changes in mitochondrial energy metabolism result in decreased cardiac efficiency. Cardiac mitochondria use oxygen to produce adenosine triphosphate (ATP), a high-energy molecule that drives numerous cellular processes, from metabolites derived from oxidation of glucose or fatty acids. Increasing fatty acid oxidation in the heart decreases glucose oxidation, and vice versa. Although glucose oxidation is a more oxygen-efficient source of energy, there is an excessive reliance on fatty acid oxidation and/or uncoupling of glycolysis from glucose oxidation in cardiac mitochondria of patients afflicted with certain types of heart disease, such as heart failure, ischemic heart disease, and diabetic cardiomyopathies. As a result, the efficiency of energy generation and production of ATP is reduced, with the corollary that the pumping capacity of the heart is reduced.

The compound 2-[4-[(2,3,4-trimethoxyphenyl)methyl]piperazin-1-yl]ethyl pyridine-3-carboxylate, referred to herein as CV-8972, was recently identified as therapeutic candidate to improve cardiac efficiency in patients with a variety of cardiovascular conditions. CV-8972 is a dual prodrug that is broken down in the body into metabolic products that fall into two different functional classes. Metabolites in the first class promote glucose oxidation by inhibiting oxidation of fatty acids and include trimetazidine and its ethylene glycol derivative 2-[4-[(2,3,4-trimethoxyphenyl)methyl]piperazin-1-yl]ethanol, referred to herein as CV-8814. These products are generated sequentially from CV-8972 catabolism: CV-8972 is initially hydrolyzed to yield CV-8814 and niacin, and the ethylene glycol moiety of CV-8814 is subsequently removed to produce trimetazidine. Niacin represents the second functional class of CV-8972 metabolites because it serves as a precursor of nicotinamide adenine dinucleotide (NAD⁺). NAD⁺ mediates electron transport in the mitochondria, thereby allowing mitochondria to derive energy from the citric acid cycle and oxidative phosphorylation. Thus, niacin increases the efficiency of energy production by cardiac mitochondria, regardless of whether the citric acid cycle is driven by products generated by oxidation of glucose or fatty acids.

An insight of the invention is that optimal therapeutic invention often requires providing members of the different functional classes of CV-8972 metabolites in different quantities. When CV-8972 is administered to a patient, however, the ratio of promoters of glucose oxidation to the NAD⁺ precursor niacin is fixed. Moreover, the ceiling of the therapeutic window of CV-8972 is determined by side effects resulting from the effective dose of niacin that the patient receives. In contrast, side effects attributable to the effective doses of CV-8814 and trimetazidine are relatively minor when CV-8972 is administered at the upper limit of tolerated doses, and patients may benefit from higher levels of CV-8814 and trimetazidine than those afforded at such doses of CV-8972. The invention solves this problem through the use of combination therapies that include administration of CV-8972 and CV-8814 as distinct compounds. Whereas metabolism of CV-8972 yields both promoters of glucose oxidation and niacin, metabolism of CV-8814 only produces the former. By providing CV-8972 and CV-8814 as separate therapeutic agents, dosing of the products that promote glucose oxidation and dosing of the NAD⁺ precursor niacin can be adjusted independently. Consequently, the combination therapies permit each of the active metabolic products of these drugs to be delivered at optimal therapeutic levels. The therapies and methods of the invention are useful for treating a wide range of cardiovascular conditions.

### Combination Therapies that Include CV-8972 and 8814

The invention provides combination therapies that include CV-8972 and CV-8814. CV-8972 has the structure of Formula (X):

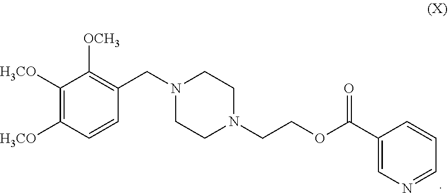 [SMILES: COc1ccc(CN2CCN(CCOC(=O)c3cccnc3)CC2)c(OC)c1OC]

When CV-8972 is administered to a subject, it is initially broken into niacin, also called nicotinic acid, and CV-8814, which has the structure of Formula (IX):

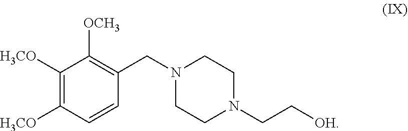 [SMILES: COc1ccc(CN2CCN(CCO)CC2)c(OC)c1OC]

CV-8814 is a hydroxyethyl derivative of trimetazidine, and the hydroxyethyl group is subsequently removed in the body to provide trimetazidine. CV-8814 is described in U.S. Pat. No. 4,100,285, and CV-8972 and its metabolic products are described in U.S. Pat. No. 10,556,013, the contents of each of which are incorporated herein by reference.

The therapeutic properties of CV-8972 are due to the effects of its various metabolic products. As indicated above, niacin serves as a precursor for synthesis of nicotinamide adenine dinucleotide (NAD⁺), the oxidized form of an essential coenzyme in the mitochondrial electron transport reaction. Supplying a NAD⁺ precursor ensures that mitochondrial redox reactions occur robustly to drive ATP synthesis, regardless of whether oxidation of glucose or fatty acids is used to feed the citric acid cycle.

The other key metabolic products of CV-8972 are CV-8814 and trimetazidine. Both CV-8814 and trimetazidine inhibit 3-ketoacyl-CoA thiolase, which is required for fatty acid oxidation. Consequently, cells are forced to rely on glucose oxidation to generate metabolites that can drive the citric acid cycle and support oxidative phosphorylation to produce ATP. Thus, both 8814 and trimetazidine are active pharmaceutical ingredients (APIs) produced by metabolism of CV-8972. However, CV-8814 does not produce the same undesirable side effects as trimetazidine. In addition, due to the sequential metabolism of CV-8972, the level of circulating trimetazidine following a dose of CV-8972 is much lower than the level following of comparable dose of trimetazidine itself. Therefore, compared to unadulterated trimetazidine, CV-8972 provides a more sustained level of circulating API and fewer side effects.

Because combination therapies of the invention include CV-8972 and CV-8814 as separate compounds, the therapies allow the relative quantities of the NAD⁺ precursor niacin and the promoters of glucose oxidation to be adjusted for optimal therapeutic invention. For example, if it is determined that a patient would benefit from a molar ratio of niacin to glucose oxidation promoters that is close to unity, i.e., only slightly less than 1:1, a combination therapy with a high ratio of CV-8972:CV-8814 is used. For many patients, however, it is advantageous to deliver a molar ratio of niacin to glucose oxidation promoters that is much lower, e.g., ≤1:2, and combination therapies with a low ratio of CV-8972:CV-8814 are appropriate for those patients. The ratio of CV-8972:CV-8814 may be expressed as a mass ratio, molar ratio, or any other suitable indicator of the relative quantities of the two compounds.

For example and without limitation, the mass ratio of the compound of formula (X) to the compound of formula (IX) may be about 10:1, about 5:1, about 2:1, about 1:1, about 1:2, about 1:5, about 1:10, about 1:20, about 1:50, or about 1:100. The mass ratio of the compound of formula (X) to the compound of formula (IX) may be from about 10:1 to about 5:1, from about 10:1 to about 2:1, from about 10:1 to about 1:1, from about 10:1 to about 1:2, from about 10:1 to about 1:5, from about 10:1 to about 1:10, from about 10:1 to about 1:20, from about 10:1 to about 1:50, from about 10:1 to about 1:100, from about 5:1 to about 2:1, from about 5:1 to about 1:1, from about 5:1 to about 1:2, from about 5:1 to about 1:5, from about 5:1 to about 1:10, from about 5:1 to about 1:20, from about 5:1 to about 1:50, from about 5:1 to about 1:100, from about 2:1 to about 1:1, from about 2:1 to about 1:2, from about 2:1 to about 1:5, from about 2:1 to about 1:10, from about 2:1 to about 1:20, from about 2:1 to about 1:50, from about 2:1 to about 1:100, from about 1:1 to about 1:2, from about 1:1 to about 1:5, from about 1:1 to about 1:10, from about 1:1 to about 1:20, from about 1:1 to about 1:50, from about 1:1 to about 1:100, from about 1:2 to about 1:5, from about 1:2 to about 1:10, from about 1:2 to about 1:20, from about 1:2 to about 1:50, from about 1:2 to about 1:100, from about 1:5 to about 1:10, from about 1:5 to about 1:20, from about 1:5 to about 1:50, from about 1:5 to about 1:100, from about 1:10 to about 1:20, from about 1:10 to about 1:50, from about 1:10 to about 1:100, from about 1:20 to about 1:50, from about 1:20 to about 1:100, or from about 1:50 to about 1:100.

Combination therapies of the invention may include defined daily dosages of CV-8972, CV8814, or both. The daily dosage of a compound indicates that amount of that compound to be provided to a subject over a 24-hour period. A daily dosage may be provided in a single dose, or it may be provided in multiple doses provided at different times over a 24-hour period. For example, a daily dosage may be provided in 2, 3, 4, 5, 6, 7, 8, or more doses. For example and without limitation, the daily dosages of the compounds of formulas (X) and (IX) may each independently be about 10 mg, about 20 mg, about 50 mg, about 100 mg, about 200 mg, about 500 mg, about 1000 mg, about 2000 mg, or about 5000 mg. The daily dosages of the compounds of formulas (X) and (IX) may each independently be from about 10 mg to about 20 mg, from about 10 mg to about 50 mg, from about 10 mg to about 100 mg, from about 10 mg to about 200 mg, from about 10 mg to about 500 mg, from about 10 mg to about 1000 mg, from about 10 mg to about 2000 mg, from about 10 mg to about 5000 mg, from about 20 mg to about 50 mg, from about 20 mg to about 100 mg, from about 20 mg to about 200 mg, from about 20 mg to about 500 mg, from about 20 mg to about 1000 mg, from about 20 mg to about 2000 mg, from about 20 mg to about 5000 mg, from about 50 mg to about 100 mg, from about 50 mg to about 200 mg, from about 50 mg to about 500 mg, from about 50 mg to about 1000 mg, from about 50 mg to about 2000 mg, from about 50 mg to about 5000 mg, from about 100 mg to about 200 mg, from about 100 mg to about 500 mg, from about 100 mg to about 1000 mg, from about 100 mg to about 2000 mg, from about 100 mg to about 5000 mg, from about 200 mg to about 500 mg, from about 200 mg to about 1000 mg, from about 200 mg to about 2000 mg, from about 200 mg to about 5000 mg, from about 500 mg to about 1000 mg, from about 500 mg to about 2000 mg, from about 500 mg to about 5000 mg, from about 1000 mg to about 2000 mg, from about 1000 mg to about 5000 mg, or from about 2000 mg to about 5000 mg.

Combination therapies may include CV-8972 and CV-8814 that are contained or provided in separate formulations. Combination therapies may include CV-8972 and CV-8814 that are contained or provided in a single formulation.

In combination therapies of the invention, each of CV-8972 and CV-8814 may independently be present as a pharmaceutically acceptable salt.

In combination therapies of the invention, each of CV-8972 and CV-8814 may independently include one or more atoms that are enriched for an isotope. For example, the compounds may have one or more hydrogen atoms replaced with deuterium or tritium. Isotopic substitution or enrichment may occur at carbon, sulfur, or phosphorus, or other atoms. The compounds may be isotopically substituted or enriched for a given atom at one or more positions within the compound, or the compounds may be isotopically substituted or enriched at all instances of a given atom within the compound.

### Pharmaceutical Compositions

The invention provides pharmaceutical compositions that contain CV-8972, CV-8814, or both. The composition may be formulated for any route or mode of administration. For example and without limitation, the composition may be formulated for buccal, dermal, enteral, intraarterial, intramuscular, intraocular, intravenous, nasal, oral, parenteral, pulmonary, rectal, subcutaneous, topical, or transdermal administration. The composition may be formulated for administration by injection or with or on an implantable medical device (e.g., stent or drug-eluting stent or balloon equivalents).

A pharmaceutical composition containing one or more the compounds may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated, or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Patent Publication No. 2003/0232877, incorporated by reference herein in their entirety.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil. An alternative oral formulation, where control of gastrointestinal tract hydrolysis of the compound is sought, can be achieved using a controlled-release formulation, where a compound is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Modified release formulations of pharmaceutical compositions may contain mixtures that include erodible polymers that promote swelling of the mixture in an aqueous environment. Pharmaceutical compositions that contain CV-8972 and one or more erodible polymers are described in co-pending, co-owned Application Nos. 63/046,115 and 63/046,117. An erodible polymer is any polymer that breaks down inside the body within a physiologically relevant time frame. The erodible polymer may have other characteristics that promote the gradual release of the modified form of trimetazidine from the mixture. For example and without limitation, the polymer may be one or more of the following: biocompatible, i.e., not harmful to living tissue; hydrophilic; hygroscopic; tending to form a hydrogel.

Without wishing to be bound by theory, the polymer-containing mixtures may promote gradual release by one or more mechanisms. For example, swelling of the mixture by absorption of water may facilitate diffusion of the modified form of trimetazidine from the mixture. Degradation of the polymer may also allow the modified form of trimetazidine to be released from the mixture. Osmotic pressure due the high concentration gradient of compound between the inside and outside of the mixture may also contribute to diffusion of the modified form of trimetazidine from the mixture.

For example and without limitation, the polymer may be a cellulose derivative, a gelatin derivative, e.g., a cross-linked gelatin derivative, or a polyester derivative.

Derivatives of cellulose, a linear chain of β(1→4) linked D-glucose units, include polymers that contain substitutions on one of more of the hydroxyl groups of each glucose unit. Substituents may be organic or inorganic and are typically attached via ester or ether linkages. Cellulose ester derivatives include carboxymethyl cellulose (CMC), e.g., sodium carboxymethyl cellulose, ethyl cellulose, ethyl hydroxyethyl cellulose, ethyl methyl cellulose, hydroxyethyl cellulose, hydroxyethyl methyl cellulose, hydroxypropyl cellulose (HPC), hydroxypropyl methylcellulose (HPMC), and methylcellulose. Cellulose ether derivatives include cellulose acetate, cellulose acetate butyrate, cellulose acetate propionate, cellulose propionate, cellulose sulfate, cellulose triacetate, and nitrocellulose. The use of cellulose-based polymers to form biodegradable hydrogels is known in the art and described in, for example, Sannino, et al., Biodegradable Cellulose-based Hydrogels: Design and Applications, Materials 2009, 2, 353-373; doi:10.3390/ma2020353, the contents of which are incorporated herein by reference.

The mixture may contain multiple polymers or multiple polymeric forms of the same polymer. For example, HPMC polymeric forms may differ in a variety of physical properties, including viscosity, degree of methoxyl substitution, degree of hydroxypropoxyl substitution, or average molecule weight.

The viscosity of a HPMC polymeric form may be determined by testing under standard conditions, including the concentration of HPMC in the solution and the temperature of the solution. For example and without limitation, the HPMC concentration may be 1%, 1.5%, 2%, 2.5%, or 3%. For example and without limitation, the temperature of the solution may be 15° C., 16° C., 17° C., 18° C., 19° C., 20° C., 21° C., 22° C., 23° C., 24° C., or 25° C.

A polymeric form of a cellulose derivative, such as HPMC, may have a defined viscosity. For example and without limitation, a polymeric form of HPMC may have a viscosity of from about 2 cP to about 4 cP, from about 4 cP to about 6 cP, from about 5 cP to about 8 cP, from about 12 cP to about 18 cP, from about 40 cP to about 60 cP, from about 80 cP to about 120 cP, from about 300 cP to about 500 cP, from about 1200 cP to about 2400 cP, from about 2500 cP to about 5000 cP, from about 9000 cP to about 18,000 cP, from about 12,000 cP to about 24,000 cP, from about 12,000 cP to about 24,000 cP, from about 75,000 cP to about 150,000 cP, at least about 2 cP at least about 4 cP at least about 5 cP at least about 12 cP at least about 40 cP at least about 80 cP at least about 300 cP at least about 1200 cP at least about 2500 cP at least about 9000 cP at least about 12,000 cP at least about 12,000 cP at least about 75,000 cP less than about 4 cP, less than about 6 cP, less than about 8 cP, less than about 18 cP, less than about 60 cP, less than about 120 cP, less than about 500 cP, less than about 2400 cP, less than about 5000 cP, less than about 18,000 cP, less than about 24,000 cP, less than about 24,000 cP, or less than about 150,000 cP for a 2% aqueous solution of the polymeric form at 20° C.

Polymeric forms of cellulose derivatives, such as HPMC, may vary in their degree of substitution of the glucose units. The degree of substitution may be expressed as a weight percentage of the substituent or as a molar ratio of substituent to glucose unit. For a cellulose derivative that has two different substituents, such as HPMC, the polymeric form may be described by the degree of substitution for each substituent.

Each polymeric form of HPMC may independently have a defined degree of methoxyl substitution. For example and without limitation, the degree of methoxyl substitution may be from about 19% to about 24%, from about 22% to about 24%, from about 27% to about 30%, from about 27% to about 30%, or from about 28% to about 32%.

Each polymeric form of HPMC may independently have a defined degree of hydroxypropoxyl substitution. For example and without limitation, the degree of hydroxypropoxyl substitution may be from about 4% to about 8%, from about 7% to about 10%, from about 7% to about 12%, from about 8% to about 10%, from about 8% to about 11%, or from about 9% to about 12%.

Each polymeric form of HPMC may independently have a defined average molecular weight. The average molecular weight may be about 10 kDa, about 13 kDa, about 20 kDa, about 26 kDa, about 41 kDa, about 63 kDa, about 86 kDa, about 110 kDa, about 120 kDa, about 140 kDa, about 180 kDa, or about 220 kDa.

When multiple forms of a polymer, such as HPMC, are present, one or more polymeric forms may be present in a defined amount. For example and without limitation, a polymer, such as HPMC, may contain about 50%, about 60%, about 70%, about 80%, about 90%, about 95%, about 96%, about 97%, about 98%, about 99%, at least 50%, at least 60%, at least 70%, at least 80%, at least 90%, at least 95%, at least 96%, at least 97%, at least 98%, or at least 99% by weight of one polymeric form.

Pharmaceutical compositions may contain a crystal form of CV-8972 or CV-8814. As described in co-pending, co-owned U.S. Application No. 63/046,120, CV-8972 may exist in at least five polymorphic forms: Form A, Form B, Form C, Form D, and Form E. A pharmaceutical composition may contain one polymorph of CV-8972 and be substantially free of one or more other polymorphs. For example, the composition may include a Form A polymorph and be substantially free of polymorphs of Form B, Form C, Form D, and Form E.

A composition containing a polymorph of CV-8972 may be substantially free of one or more other polymorphic forms of CV-8972 if the composition contains the predominant polymorph at a defined level of purity. Purity may be expressed as the amount of predominant polymorph as a percentage of the total weight of two of more polymorphs of CV-8972.

In certain embodiments, the total weight is the weight of all polymorphs of CV-8972 in the composition. For example, a composition that contains the Form A polymorph and is substantially free of other polymorphs may contain Form A at a defined weight percentage of all polymorphs of CV-8972 in the composition. For example, the composition may contain Form A at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of all polymorphs of CV-8972 in the composition.

In certain embodiments, the total weight is the weight of selected polymorphs of CV-8972 in the composition. For example, a composition that contains the Form A polymorph and is substantially free of the Form B polymorph may contain Form A at a defined weight percentage of Forms A and B. For example, the composition may contain Form A at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of Forms A and B of CV-8972 in the composition. Similarly, a composition that contains the Form A polymorph and is substantially free of the Form B and C polymorphs may contain Form A at a defined weight percentage of Forms A, B, and C. For example, the composition may contain Form A at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of Forms A, B, and C of CV-8972 in the composition.

Alternatively or additionally, a composition containing a polymorph of CV-8972 may be substantially free of one or more other polymorphic forms of CV-8972 if the composition contains the secondary polymorphs at levels below a defined level. Presence of a secondary polymorphs may be defined as the amount of one or more secondary polymorphs as a percentage of the total weight of two of more polymorphs of CV-8972.

In certain embodiments, the total weight is the weight of all polymorphs of CV-8972 in the composition. For example, a composition that contains the Form A polymorph and is substantially free of other polymorphs may contain all polymorphs other than Form A at a defined weight percentage of all polymorphs of CV-8972 in the composition. For example, the composition may contain all polymorphs other than Form A at below 5% by weight, below 4% by weight, below 3% by weight, below 2% by weight, below 1% by weight, below 0.5% by weight, below 0.4% by weight, below 0.3% by weight, below 0.2% by weight, or below 0.1% by weight of all polymorphs of CV-8972 in the composition.

In certain embodiments, the total weight is the weight of selected polymorphs of CV-8972 in the composition. For example, a composition that contains the Form A polymorph and is substantially free of the Form B polymorph may contain Form B at a defined weight percentage of Forms A and B. For example, the composition may contain Form B at below 5% by weight, below 4% by weight, below 3% by weight, below 2% by weight, below 1% by weight, below 0.5% by weight, below 0.4% by weight, below 0.3% by weight, below 0.2% by weight, or below 0.1% by weight of Forms A and B of CV-8972 in the composition. Similarly, a composition that contains the Form A polymorph and is substantially free of the Form B and Form C polymorphs may contain Forms B and C at a defined weight percentage of Forms A, B, and C. For example, the composition may contain Forms B and C at below 5% by weight, below 4% by weight, below 3% by weight, below 2% by weight, below 1% by weight, below 0.5% by weight, below 0.4% by weight, below 0.3% by weight, below 0.2% by weight, or below 0.1% by weight of Forms A, B, and C of CV-8972 in the composition.

The crystal may contain a salt form of CV-8972. For example, the Form A polymorph CV-8972 is a trihydrochloride salt. Thus, the composition may include CV-8972 and the chloride ion a defined stoichiometric ratio. The composition may include CV-8972 and the chloride ion in a 1:3 stoichiometric ratio.

The crystal may contain a hydrated form of CV-8972. For example, the Form A polymorph CV-8972 is a monohydrate. Thus, the composition may include a monohydrate form of CV-8972, such as the Form A polymorph. The composition may include an anhydrous form of CV-8972, such as a Form B, Form D, or Form E polymorph.

The pharmaceutical composition may be formulated as a single unit dosage. The pharmaceutical composition may be formulated as divided dosages.

The composition may contain a defined amount of CV-8972 or CV-8814. The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of CV-8972 or CV-8814. The dose may contain about 10 mg, about 25 mg, about 50 mg, about 100 mg, about 200 mg, about 300 mg, or about 400 mg of CV-8972 or CV-8814.

### Providing a Compound to a Subject

The invention provides methods of treating diseases, disorders, and conditions by providing both CV-8972 and CV-8814 to a subject having a disease, disorder, or condition. Each of CV-8972 and CV-8814 may independently be provided by any suitable route or mode of administration. For example and without limitation, each compound may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents).

Each of CV-8972 and CV-8814 may independently be provided according to a dosing regimen. A dosing regimen may include a dosage, a dosing frequency, or both.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose or dosage may contain a defined amount of CV-8972 or CV-8814 that improves cardiac mitochondrial function, such as any of the doses described above in relation to pharmaceutical compositions containing CV-8972 or CV-8814.

The dose or dosage may be provided in a single unit, i.e., the dose may be provided as a single tablet, capsule, pill, etc. Alternatively, the dose or dosage may be provided in multiple units, i.e., the dose or dosage may be provided as multiple tablets, capsules, pills, etc.

The dosing may continue for a defined period or may continue indefinitely. For example and without limitation, doses may be provided for at least one week, at least two weeks, at least three weeks, at least four weeks, at least six weeks, at least eight weeks, at least ten weeks, at least twelve weeks or more. In the context of prophylaxis, the dosing may precede a planned intervention.

### Diseases, Disorders, and Conditions

The combination therapies, methods of treatment, and pharmaceutical compositions of the invention may be used to treat a disease, disorder, or condition in a subject. The disease, disorder, or condition may be any condition that can be ameliorated by improving cardiac mitochondrial function. The disease, disorder, or condition may be a cardiovascular condition. The disease, disorder, or condition may be acute coronary syndrome, acute heart failure, advanced heart failure, aneurysm, angina, anthracycline-induced cardiotoxicity, atherosclerosis, cardiac allograft vasculopathy, cardiac steatosis, cardiac transplant vasculopathy, cardiomyopathy, cerebral vascular disease, chronic coronary syndrome, chronic heart failure, congenital heart disease, contrast nephropathy, coronary artery disease (CAD), coronary heart disease, diabetic cardiomyopathy, dilated cardiomyopathy (DCM, including idiopathic, heart attack, heart disease, heart failure with mildly reduced ejection fraction (HFmrEF), heart failure with preserved ejection fraction (HFpEF), heart failure with reduced ejection fraction (HFrEF), heart failure, hibernating myocardium, high blood pressure (hypertension), hypertrophic cardiomyopathy (HCM, including non-obstructive or obstructive), intermittent claudication, ischemia with non-obstructive coronary arteries (INOCA), ischemia-reperfusion injury, ischemic cardiomyopathy, ischemic heart disease, microvascular angina, myocardial dysfunction induced by anti-cancer drugs, myocardial infarction with non-obstructive coronary arteries (MINOCA), myocarditis, non-familial and familial/genetic), non-ischemic cardiomyopathy, pericardial disease, peripartum cardiomyopathy, peripheral arterial disease, peripheral vascular disease, pulmonary arterial hypertension, pulmonary hypertension, refractory angina, restrictive cardiomyopathy, rheumatic heart disease, right heart failure, right ventricular failure, stable angina, stroke, stunned myocardium, tachycardiomyopathy, Takotsubo cardiomyopathy, transient ischemic attack, unstable angina, or valvular heart disease.

Angina pectoris (angina) is chest pain or pressure that is typically due to insufficient blood flow to the heart muscle. The pain or discomfort is retrosternal or left-sided and may radiate to the left arm, neck, jaw, or back. Several classifications of angina are known. Stable angina, also called effort angina, is related to myocardial ischemia. In stable angina, chest discomfort and associated symptoms are usually triggered by some physical activity, such as running or walking, but symptoms are minimal or non-existent when the patient is at rest or has taken sublingual nitroglycerin. Symptoms typically abate several minutes after activity and recur when activity resumes. Symptoms may also be induced by cold weather, heavy meals, and emotional stress. Unstable angina is angina that changes or worsens. Unstable angina has at least one of the following features: (1) it occurs at rest or with minimal exertion, usually lasting more than 10 minutes, (2) it is severe and of new onset, i.e., within the prior 4-6 weeks, and (3) it occurs with a crescendo pattern, i.e., distinctly more severe, prolonged, or frequent than before. Cardiac syndrome X, also called microvascular angina, is angina-like chest pain, in the context of normal epicardial coronary arteries on angiography. Its primary cause is unknown, but factors apparently involved are endothelial dysfunction and reduced flow in the tiny resistance blood vessels of the heart. Microvascular angina may be part of the pathophysiology of ischemic heart disease. Refractory angina is a chronic condition (≥3 months in duration) in which angina (1) occurs in the context of coronary artery disease (CAD), (2) cannot be controlled by a combination of optimal medical therapy, angioplasty, or bypass surgery, and (3) in which reversible myocardial ischemia has been clinically established to be the cause of the symptoms.

The subject having the disease, disorder, or condition may fall into a particular class of subjects. For example and without limitation, the subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a hospital, a field hospital, an out-of-hospital field setting, a standard office setting, community clinic, or community healthcare setting.

### EXAMPLES

The invention may be better understood in view of the examples below, which are provided for illustrative purposes only and do not limit the scope of the invention. Throughout the examples and the accompanying figures, the following synonyms for compounds of the invention may be used: CV-8972 may alternately be referred to as IMB-101 or IMB-1018972; and CV-8814 may alternately be referred to as IMB-102 or IMB-1028814.

### Example 1

### Study Overview

The effects of CV-8972, a partial fatty acid oxidation inhibitor, on glucose and fatty acid metabolism were investigated using non-invasive positron emission tomography (PET) imaging. The PET radiotracer ¹⁸F fluorodeoxyglucose (FDG), a radiolabeled glucose analog, was used to evaluate cardiac glucose metabolism, and the PET radiotracer ¹⁸F fluoro-6-thia-heptadecanoic acid (FTHA), a radiolabeled long-chain fatty acid (LCFA) analog, was used to evaluate fatty acid beta oxidation. Both radiotracers are “entrapped” in cells in a way that is proportional to either glucose or fatty acid metabolism. For example, high ¹⁸F-FDG or ¹⁸F-FTHA signal indicates high glucose or fatty acid metabolism, respectively, while, conversely, diminished uptake indicates lower metabolism.

PET imaging with ¹⁸F-FDG and ¹⁸F-FTHA was performed in healthy rats. PET images were acquired dynamically for 120 minutes after tracer injection to capture dynamic changes in cardiac glucose and fatty acid metabolism. For each tracer, animals were imaged twice, once after injection of vehicle (phosphate buffered saline, PBS) or CV-8972 (80 mg/3 mL/kg), subcutaneously injected to the back of animals 15 min prior to PET imaging.

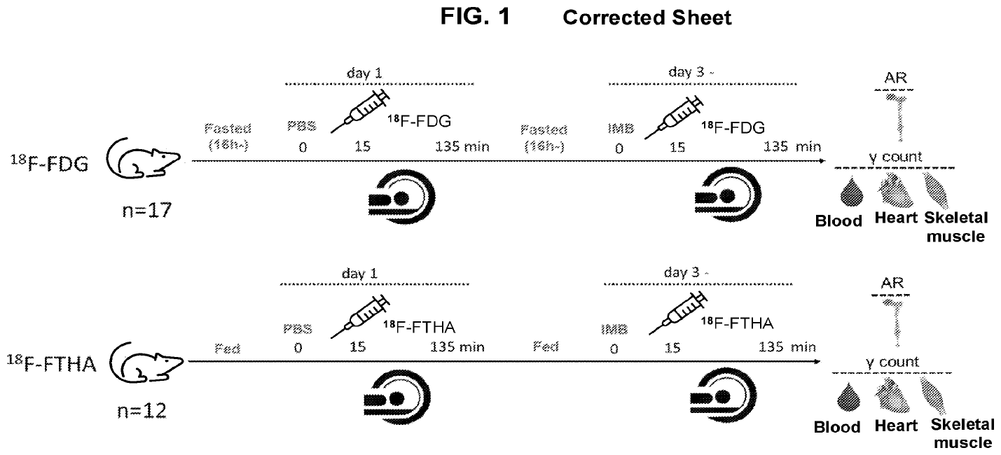

FIG. 1 is a schematic of the positron emission tomography (PET) imaging study design used to monitor the uptake of ¹⁸F-fluorodeoxyglucose (¹⁸F-FDG) and ¹⁸F-14(R,S)-(18)F-fluoro-6-thia-heptadecanoic acid (¹⁸F-FTHA) into heart tissue of rats in response to treatment with CV-8972. To monitor uptake of ¹⁸F-FDG, rats were fasted for 16 hours prior to day 1. On day 1, rats were given phosphate-buffered saline (PBS) at time 0, and 15 minutes later ¹⁸F-FDG was administered and traced in vivo by PET for two hours following administration. Rats were again fasted for 16 hours prior to day 3. On day 3, rats were given CV-8972 at time 0, and 15 minutes later ¹⁸F-FDG was administered and traced in vivo by PET for two hours following administration. Immediately following the second PET scan, the animals were sacrificed, and the levels of gamma radioactivity in the blood, heart, and skeletal muscle were measured. To monitor uptake of ¹⁸F-FTHA, rats were allowed to eat prior to day 1. On day 1, rats were given PBS at time 0, and 15 minutes later ¹⁸F-FTHA was administered and traced in vivo by PET for two hours following administration. Rats were allowed to eat prior to day 3. On day 3, rats were given CV-8972 at time 0, and 15 minutes later ¹⁸F-FTHA was administered and traced in vivo by PET for two hours following administration. Immediately following the second PET scan, the animals were sacrificed, and the levels of gamma radioactivity in the blood, heart, and skeletal muscle were measured.

### Materials and Methods

### Animals

Twenty-three female Crl:CD(SD) rats (average weight 408±46.5 g at the first scan) were purchased from Charles River Laboratories (MA, USA). Rats were housed in a temperature-controlled animal room with a 12 h-light (from 07:00 to 19:00) and 12 h-dark cycle. All animals were acclimated at least 3 days prior to use for the current experiment. All animal experiments were approved by Institutional Animal Care and Use Committee (IACUC) of the Icahn School of Medicine at Mount Sinai.

### Dynamic PET/CT Acquisition

Dynamic positron emission tomography/computed tomography (PET/CT) imaging was obtained with a Mediso nanoScan 122S PET/CT system. Cardiac glucose and fatty acid metabolisms were visualized by tracking ¹⁸F-FDG (NCM-USA LLC, NY, USA) and ¹⁸F-FTHA (Department of Radiology, New York University School of Medicine, NY, USA), respectively. Before the ¹⁸F-FDG scan, rats were fasted overnight for at least 16 hours to minimize the physiological uptake of the radiotracer to cardiomyocyte. Either vehicle (phosphate buffered saline) or CV-8972 (80 mg/3 mL/kg) was subcutaneously injected to the back of animals 15 min prior to PET imaging. Animals were then placed on the scanner, and high-resolution CT images was obtained. Following, dynamic PET was started at the same time as the injection of ¹⁸F-FDG (37 MBq, 1 mCi/0.5 mL/body) via tail vein and recorded for 120 min. Each animal underwent 2 scan sessions, one after vehicle injection and another after CV-8972 injection, with 2 or more days interval (average 2.9 days, 2-6 days). The order of the 2 imaging sessions was randomized among rats. For ¹⁸F-FTHA (37 MBq, 1 mCi/0.5 mL/body), animals received 2 scan sessions under same conditions as those for ¹⁸F-FDG except for fed condition. Among the animals which received ¹⁸F-FDG scan, 6 animals also underwent ¹⁸F-FTHA scan with more than 3 days interval (average 6.0 days, 2-14 days). Other rats underwent either ¹⁸F-FDG scan or ¹⁸F-FTHA scan only.

### Dynamic PET/CT Reconstruction and Analysis

PET images were reconstructed using a 3D ordered-subset expectation maximization/maximum a posteriori (3D-OSEM/MAP). Voxel count rates in the reconstructed images were decay corrected and converted to a standardized uptake value (SUV) by system calibration factor from a cylindrical phantom. A 3D region of interest (ROI) for the heart (left ventricle), muscle (right brachium), and blood (abdominal aorta) was drawn using AMIDE^{x)} software based on a guide by co-registered anatomic CT images to yield organ activity normalized by the body weight (g) and the residual activity at scan. From the whole acquisition, dynamic frames were calculated with the following frame rate, for both radiotracers: 8 frames at 15 sec/frame, 6 frames for 30 sec/frame, 3 frames for 300 sec/frame, and 10 frames for 600 sec/frame. Dynamic curves were analyzed using PMOD software, using a 2-compartment model for ¹⁸F-FDG and a 3-compartment model for ¹⁸F-FTHA.

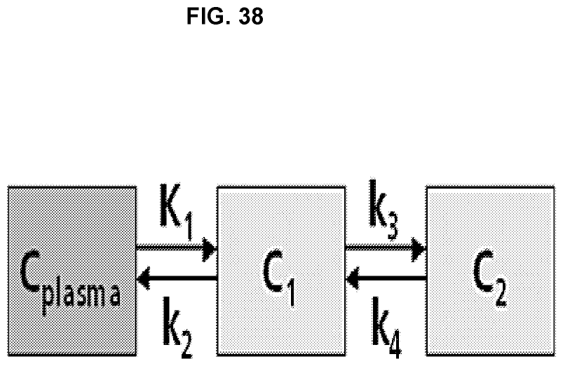

FIG. 38 shows a two-compartment model used to evaluate FDG update. Kᵢ, K₁, K₂, K₃, and K₄ were calculated as follows:

$${k}_{i} = \frac{{k}_{1} \cdot {k}_{3}}{{k}_{2} + {k}_{3}}$$

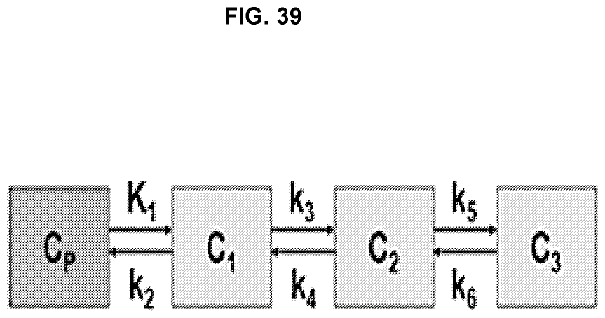

FIG. 39 shows a three-compartment model used to evaluate FTHA uptake. Kᵢ, K₁, K₂, K₃, K₄, K₅, K₆, V₁, and V₂ were calculated as follows:

$${k}_{i} = \frac{{k}_{1} \cdot {k}_{3} \cdot {k}_{5}}{{k}_{2} \cdot {k}_{4} + {k}_{2} \cdot {k}_{5} + {k}_{3} \cdot {k}_{5}}$$

$${V}_{1} = {k}_{1} \frac{{k}_{4} + {k}_{5}}{{k}_{2} \cdot {k}_{4} + {k}_{2} \cdot {k}_{5} + {k}_{3} \cdot {k}_{5}}$$

$${V}_{2} = {k}_{1} \frac{{k}_{1} \cdot {k}_{3}}{{k}_{2} \cdot {k}_{4} + {k}_{2} \cdot {k}_{5} + {k}_{3} \cdot {k}_{5}}$$

### Ex vivo Validation of the in vivo PET Signal with Gamma Counting

Shortly after the final PET/CT scan, blood was taken from abdominal vein under anesthesia with isoflurane (3-5%), and then animals were euthanized by exsanguination. After thorough perfusion with PBS, the heart and skeletal muscle (right brachium) were harvested. Radioactivity in the heart, muscle, and blood was determined with a Wizard2 2480 automatic gamma counter (Perkin Elmer, Waltham, MA) and % ID/g was calculated for each organ.

### Statistical Analysis

Results are expressed as mean±standard deviation (SD). Differences between control and CV-8972 groups were evaluated by two-tailed paired t-tests (scan data) or student t-test (ex vivo data). These analyses were performed using GraphPad Prism (GraphPad Software Inc., CA, USA) or Microsoft® Office Excel 2010 (Microsoft Corporation, WA, USA). A p value of <0.05 was classified as a statistically significant change.

### Results

### Impact of CV-8972 on Cardiac Glucose and Fatty Acid Metabolism: ¹⁸F-FDG and ¹⁸F-FTHA in vivo PET Uptake

To evaluate the effect of CV-8972 on cardiac glucose and fatty acid metabolism, the time-course of ¹⁸F-FDG and ¹⁸F-FTHA uptake (measured as SUV) was monitored in the cardiac muscle. Radiotracers uptake in the skeletal muscle and in the blood was also monitored.

In a first analysis, single corresponding dynamic frames were compared between treated and PBS injected animals.

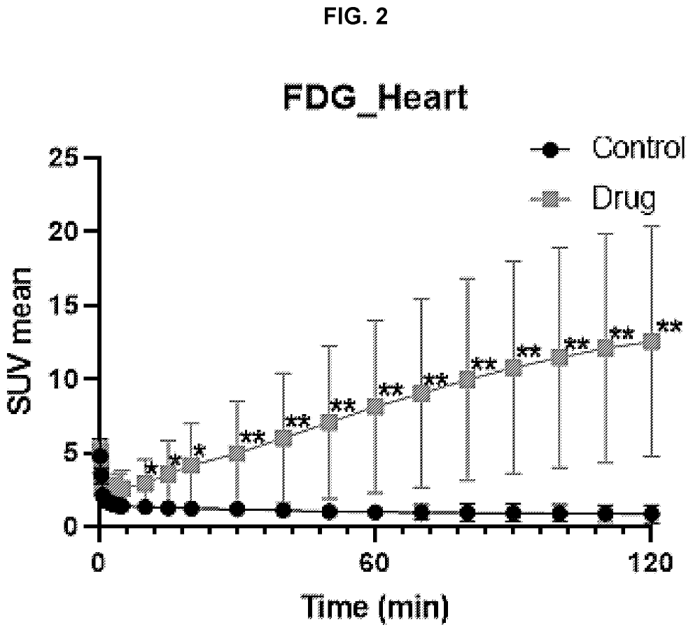

FIG. 2 is a graph showing the mean SUV of FDG in the heart at various time points following administration of either saline (Control, black circles) or CV-8972 (Drug, red squares) to rats. * p<0.05, ** p<0.01 by paired t-test.

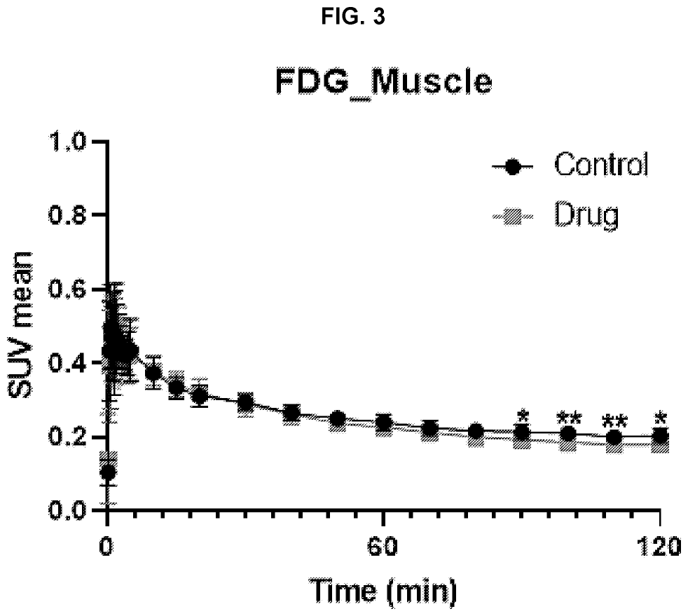

FIG. 3 is a graph showing the mean SUV of FDG in skeletal muscle at various time points following administration of saline (Control, black circles) or CV-8972 (Drug, red squares) to rats. * p<0.05, ** p<0.01 by paired t-test.

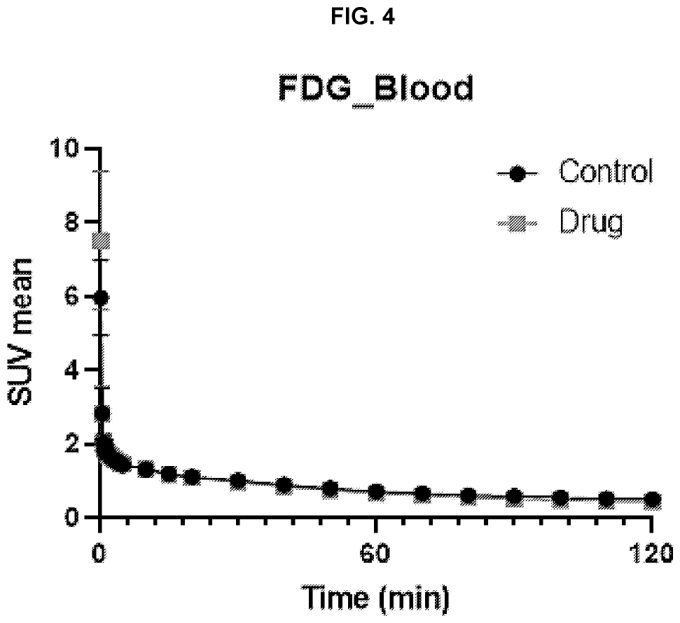

FIG. 4 is a graph showing the mean SUV of FDG in the blood at various time points following administration of either saline (Control, black circles) or CV-8972 (Drug, red squares) to rats.

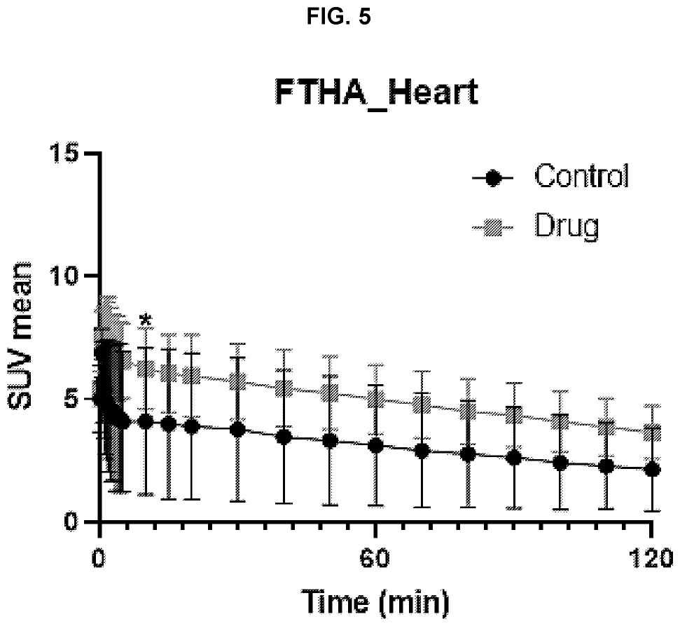

FIG. 5 is a graph showing the mean SUV of FTHA in the heart at various time points following administration of either saline (Control, black circles) or CV-8972 (Drug, red squares) to rats. * p<0.05 by paired t-test.

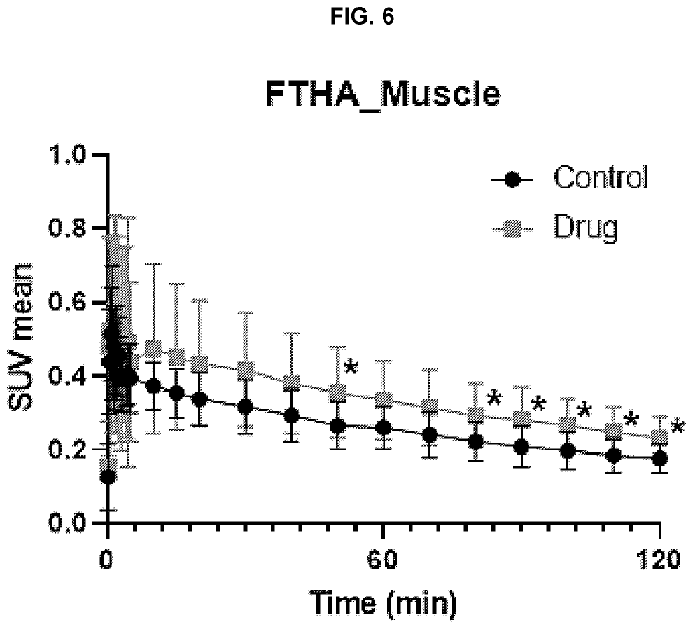

FIG. 6 is a graph showing the mean SUV of FTHA in skeletal muscle at various time points following administration of either saline (Control, black circles) or CV-8972 (Drug, red squares) to rats. * p<0.05 by paired t-test.

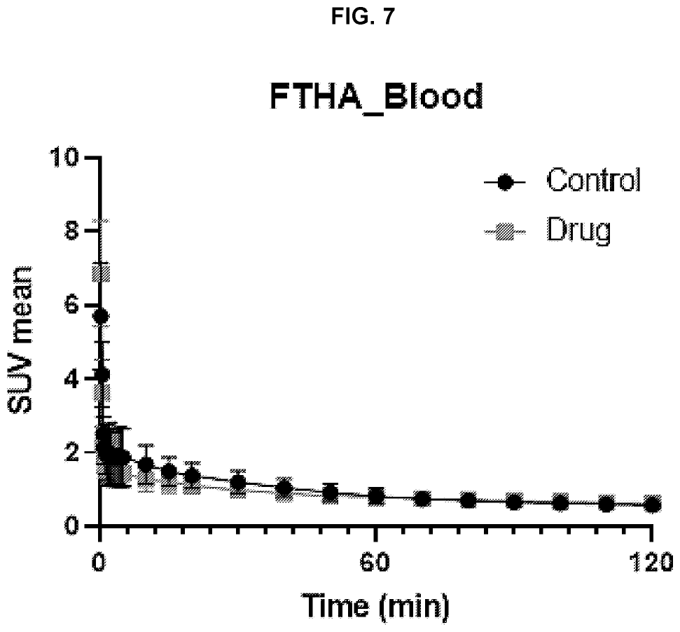

FIG. 7 is a graph showing the mean SUV of FTHA in the blood at various time points following administration of either saline (Control, black circles) or CV-8972 (Drug, red squares) to rats.

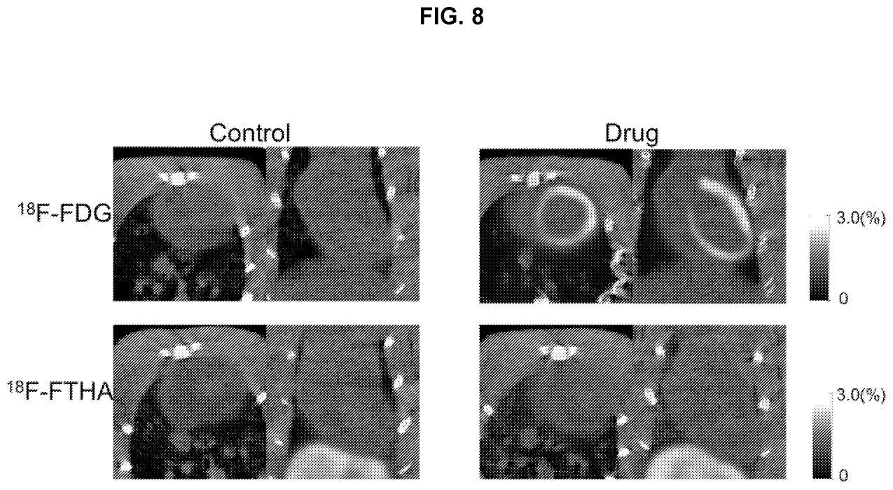

FIG. 8 shows PET/CT images FDG and FTHA following administration of either saline (Control) or CV-8972 (Drug) to rats. Images were created by overlaying the sum of PET images obtained from 0-120 min on the CT images. Each panel includes a left image that represents the short axis of the heart and a right image that represents the long axis of the heart.

This analysis overall shows that a single injection of CV-8972 significantly increased cardiac ¹⁸F-FDG uptake compared with PBS injection, indicating increased glucose uptake and metabolism after CV-8972 injection. The differences in SUV in the heart between control and CV-8972-injected animals became much more prominent over time, towards the end of the 120 min dynamic PET acquisition. The average SUV in the heart of CV-8972-injected animals at 120 min was 12.56±7.82, which is approximately 14-fold higher than that of control group (0.89±0.62, p<0.01). On the other hand, no statistically significant changes were detected in the heart when ¹⁸F-FTHA was injected, although slightly higher uptake was constantly noted in CV-8972-injected animals throughout the scan acquisition period. Similarly, slightly higher uptake of ¹⁸F-FTHA was also observed in the skeletal muscle while ¹⁸F-FDG was slightly lower. When looking at single dynamic frames, CV-8972 injection did not clearly affect ¹⁸F-FDG and ¹⁸F-FTHA signal in the blood.

In a second analysis, SUV values were calculated from one single frame representing the last 30 minutes of the PET acquisition (90-120 minutes).

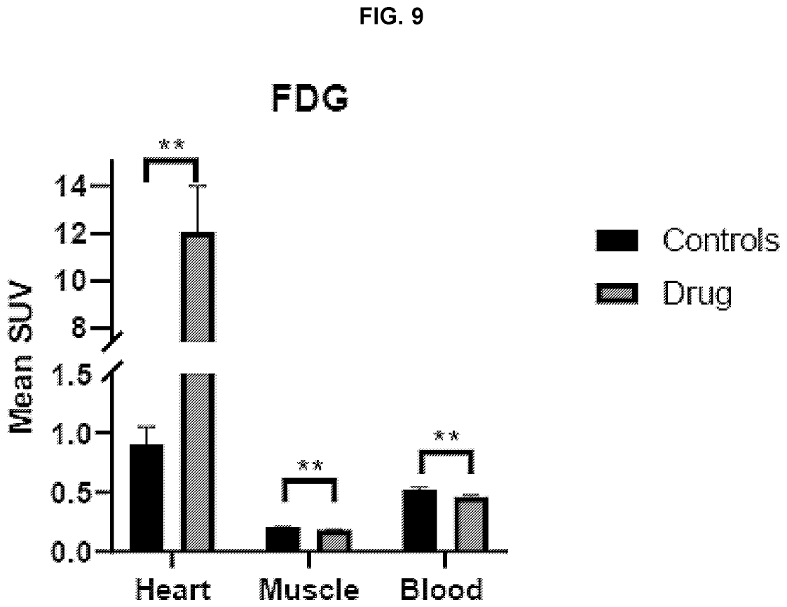

FIG. 9 is a graph showing the mean SUV of FDG in the heart, skeletal muscle, and blood in the last 30 minutes of the dynamic acquisition following administration of either saline (Control, black bars) or CV-8972 (Drug, red bars) to rats. **p<0.01 by paired t-test.

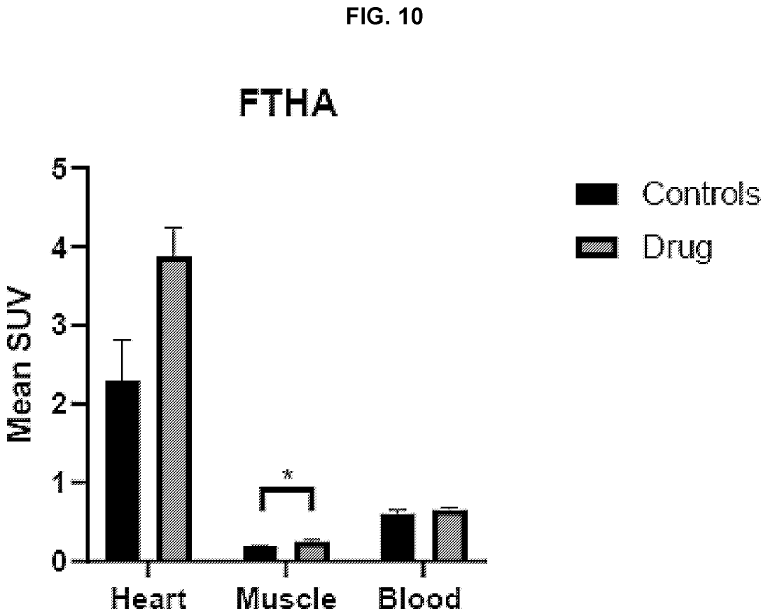

FIG. 10 is a graph showing the mean SUV of FTHA in the heart, skeletal muscle, and blood in the last 30 minutes of the dynamic following administration of either saline (Control, black bars) or CV-8972 (Drug, red bars) to rats. * p<0.05 by paired t-test.

This analysis confirmed our previous analysis on single dynamic frames. Briefly ¹⁸F-FDG uptake was significantly higher in the myocardium, but showed slightly lower signal (although reaching statistical significance) in the skeletal muscle and blood of CV-8972-injected animals. For ¹⁸F-FTHA, myocardial signal in CV-8972 animals was slightly higher without reaching statistical significance, while slightly higher signal in the skeletal muscle reached statistical significance. ¹⁸F-FTHA blood signal was unaffected by CV-8972 injection.

In a third analysis, radiotracers uptake curves were analyzed using kinetic modeling.

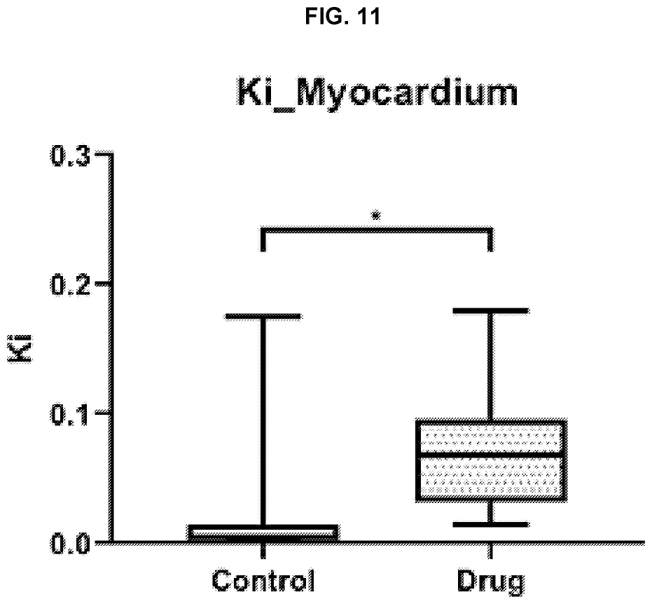

FIG. 11 is a graph of FDG uptake in the myocardium following administration of either saline (Control) or CV-8972 (Drug) to rats. Kᵢ values were determined using a two-compartment model. * p<0.05 by paired t-test.

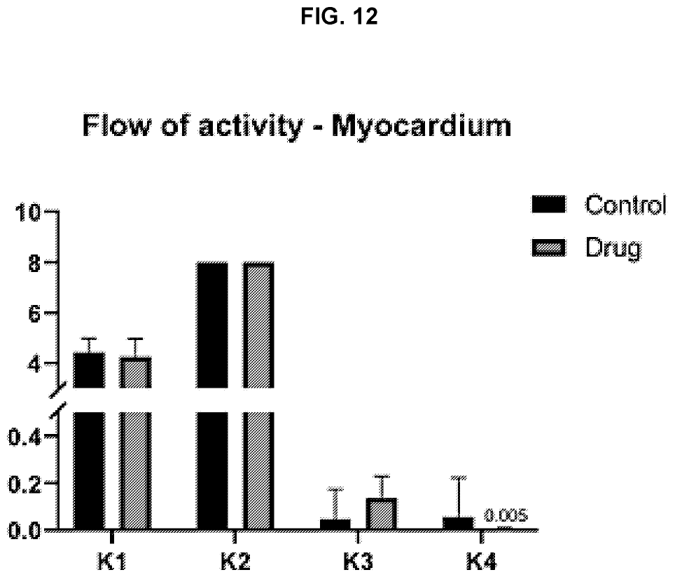

FIG. 12 is a graph of flow of activity of FDG in the myocardium following administration of either saline (Control, black bars) or CV-8972 (Drug, red bars) to rats. K₁, K₂, K₃, and K₄ values were determined using a two-compartment model.

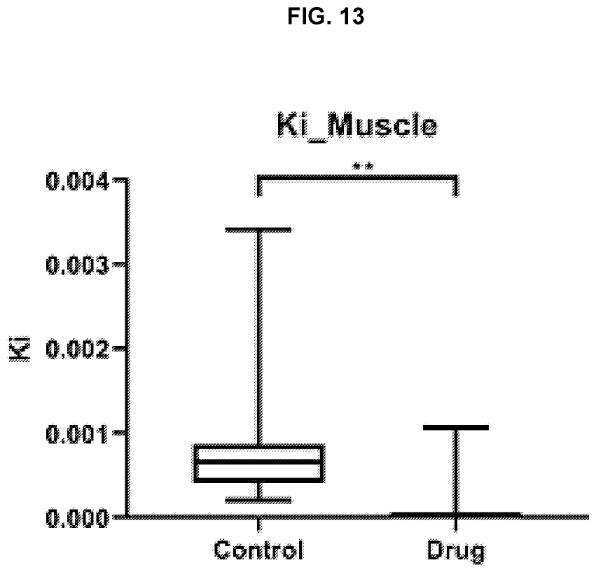

FIG. 13 is a graph of FDG uptake in skeletal muscle following administration of either saline (Control) or CV-8972 (Drug) to rats. Kᵢ values were determined using a two-compartment model. ** p<0.01 by paired t-test.

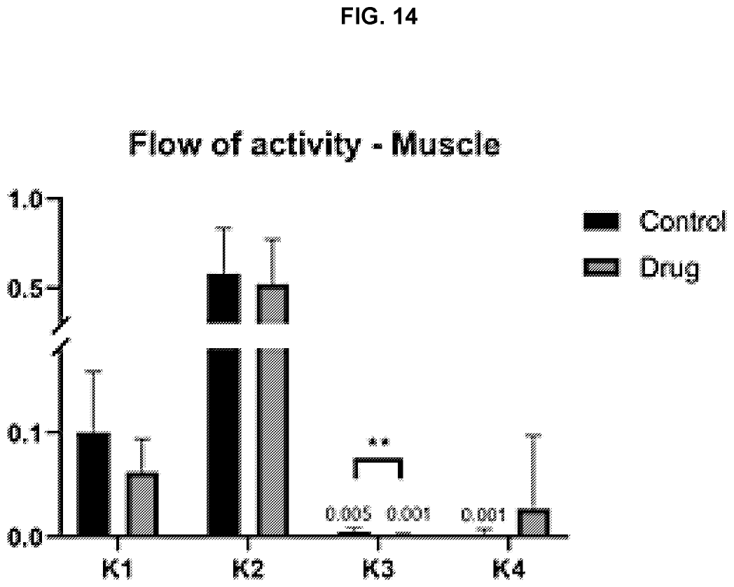

FIG. 14 is a graph of flow of activity of FDG in skeletal muscle following administration of either saline (Control, black bars) or CV-8972 (Drug, red bars) to rats. K₁, K₂, K₃, and K₄ values were determined using a two-compartment model. ** p<0.01 by paired t-test.

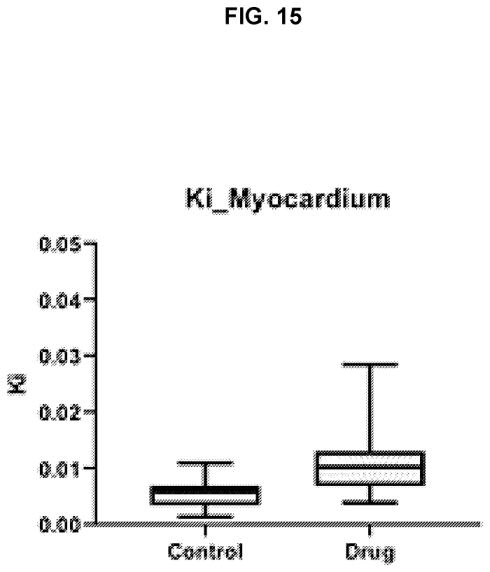

FIG. 15 is a graph of FTHA uptake in the myocardium following administration of either saline (Control) or CV-8972 (Drug) to rats. Kᵢ values were determined using a three-compartment model.

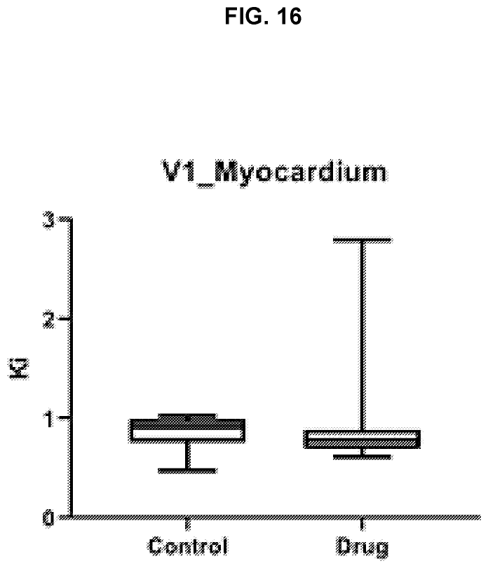

FIG. 16 is a graph of pharmacokinetic parameter V₁ of FTHA uptake in the myocardium following administration of either saline (Control) or CV-8972 (Drug) to rats. V₁ values were determined using a three-compartment model.

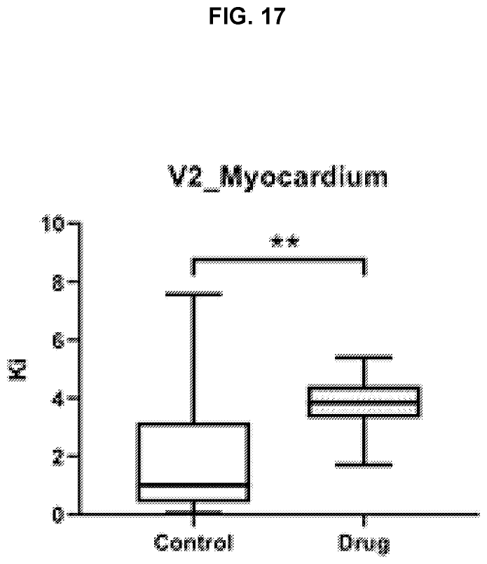

FIG. 17 is a graph of pharmacokinetic parameter V₂ of FTHA uptake in the myocardium following administration of either saline (Control) or CV-8972 (Drug) to rats. V₂ values were determined using a three-compartment model. ** p<0.01 by paired t-test.

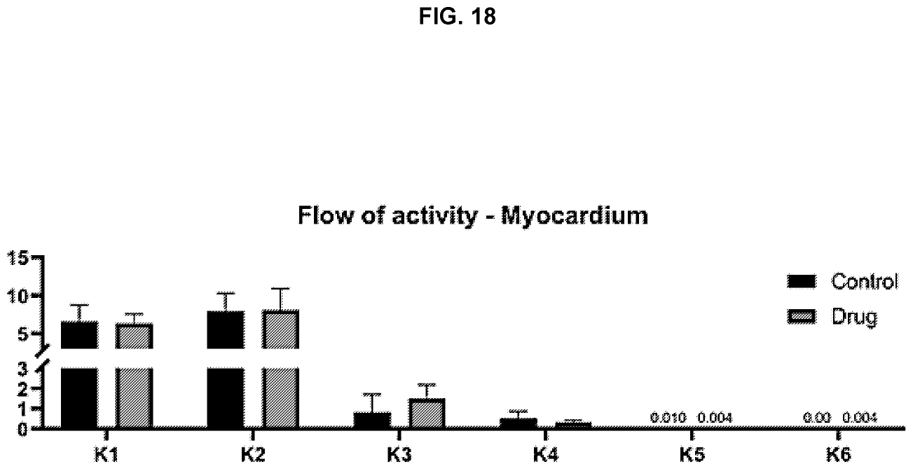

FIG. 18 is a graph of flow of activity of FTHA in the myocardium following administration of either saline (Control, black bars) or CV-8972 (Drug, red bars) to rats. K₁, K₂, K₃, K₄, K₅, and K₆ values were determined using a three-compartment model.

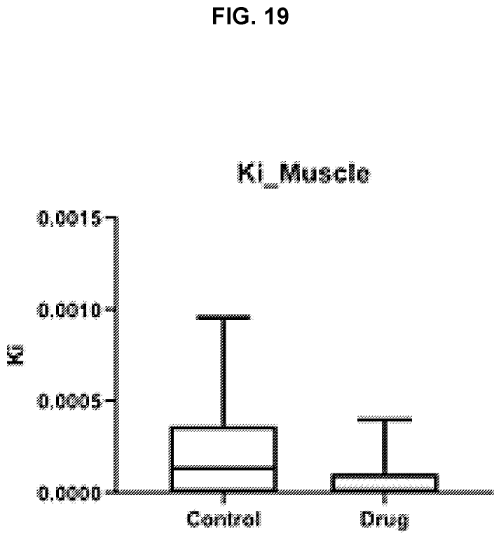

FIG. 19 is a graph of FTHA uptake in skeletal muscle following administration of either saline (Control) or CV-8972 (Drug) to rats. Kᵢ values were determined using a three-compartment model.

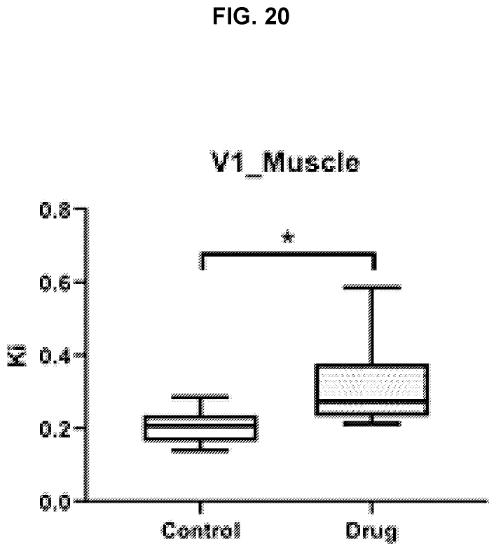

FIG. 20 is a graph of pharmacokinetic parameter V₁ of FTHA uptake in skeletal muscle following administration of either saline (Control) or CV-8972 (Drug) to rats. V₁ values were determined using a three-compartment model. * p<0.05 by paired t-test.

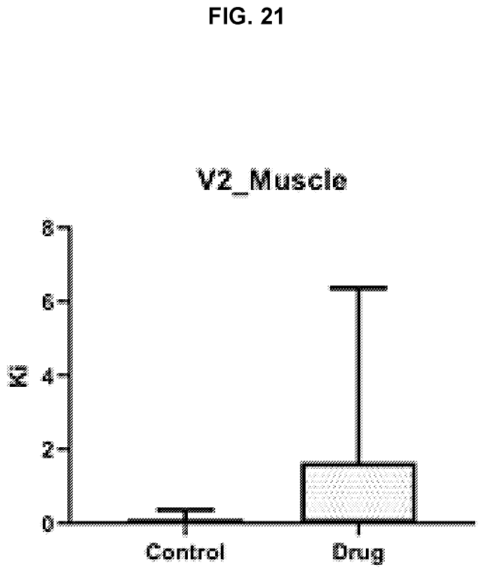

FIG. 21 is a graph of pharmacokinetic parameter V₂ of FTHA uptake in skeletal muscle following administration of either saline (Control) or CV-8972 (Drug) to rats. V₂ values were determined using a three-compartment model.

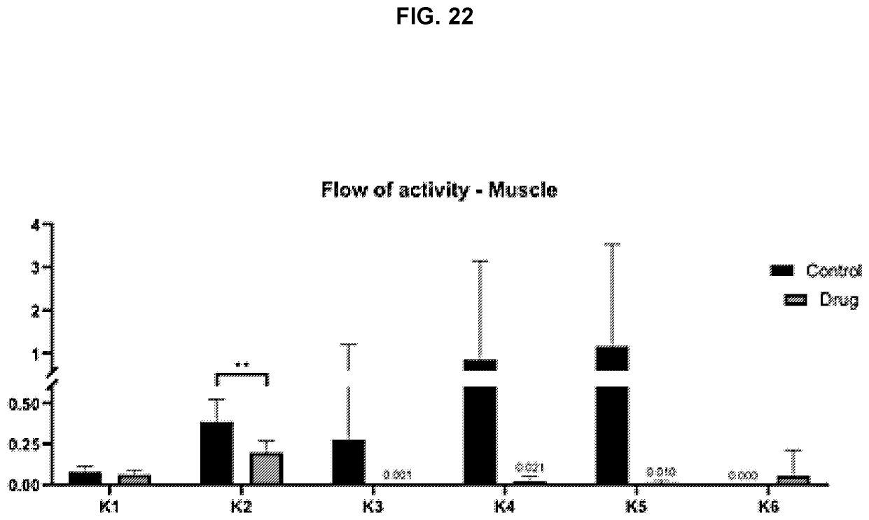

FIG. 22 is a graph of flow of activity of FTHA in skeletal muscle following administration of either saline (Control, black bars) or CV-8972 (Drug, red bars) to rats. K₁, K₂, K₃, K₄, K₅, and K₆ values were determined using a three-compartment model. ** p<0.01 by paired t-test.

Kinetic modeling of ¹⁸F-FDG confirmed overall significantly higher myocardial uptake of this tracer in CV-8972-injected animals, and lower in the skeletal muscle. Kinetic modeling of ¹⁸F-FTHA confirmed overall significantly higher uptake of this tracer into the myocardium and skeletal muscle in CV-8972-injected animals.

### Ex Vivo Quantification of Radiotracer Uptake by Gamma Counting

The radioactivity in the heart, skeletal muscle, and blood was also determined with a gamma counter shortly after PET/CT imaging for further ex vivo validation.

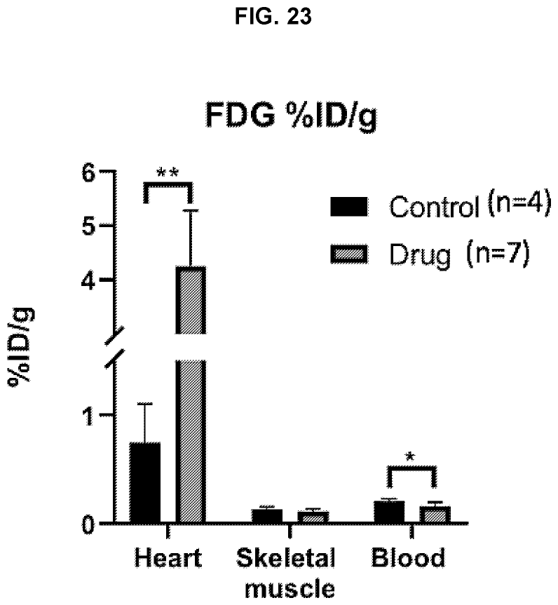

FIG. 23 is a graph of gamma radioactivity (expressed as % injected dose/g, % ID/g) from FDG in the heart, skeletal muscle, and blood following administration of either saline (Control, black bars) or CV-8972 (Drug, red bars) to rats. Data are from four animals that received saline and seven animals that received CV-8972. * p<0.05, ** p<0.01 by student t-test.

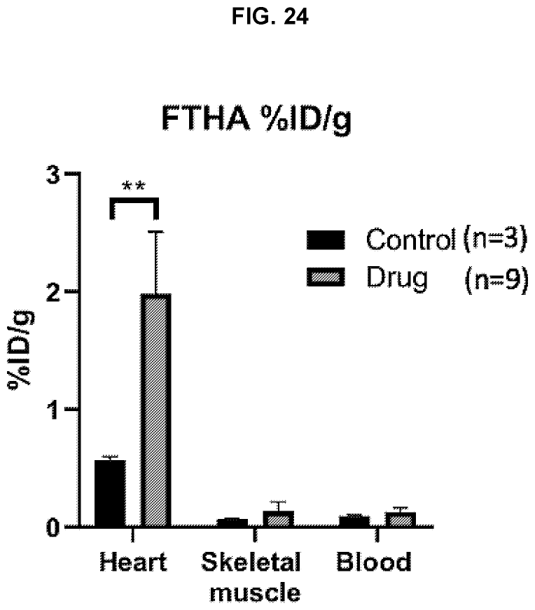

FIG. 24 is a graph of gamma radioactivity from FTHA in the heart, skeletal muscle, and blood following administration of either saline (Control, black bars) or CV-8972 (Drug, red bars) to rats. Data are from three animals that received saline and nine animals that received CV-8972. * p<0.05 by student t-test.

The mean radioactivity of the heart after CV-8972 injection was 4.25±1.03% ID/g for ¹⁸F-FDG and 1.98±0.53% ID/g for ¹⁸F-FTHA, which were 5.6- and 3.6-fold higher than the control radioactivity, respectively. The radioactivity of skeletal muscle and blood was much lower than that of the heart and did not show clear differences between control and CV-8972-injected animals.

### Conclusion

In the study described in Example 1, non-invasive in vivo imaging with micro PET/CT was performed in rats to quantify changes in glucose and fatty acid metabolism on the heart after a single injection of CV-8972. The results demonstrate a significant increase in glucose metabolism in the cardiac muscle as demonstrated by higher ¹⁸F-FDG uptake after CV-8972 injection. ¹⁸F-FTHA (a fatty acid analog) showed slightly higher uptake in the myocardium of healthy animals. These results were demonstrated by 3 different analyses of in vivo PET data, as well as ex vivo gamma counting. In particular, ¹⁸F-FDG uptake differences could be detected by static analysis of PET images between 90 and 120 minutes after radiotracer injection, indicating the feasibility of this approach for evaluating the drug effect in humans using validated static in vivo PET imaging.

Results show that CV-8972 administration increased rat heart ¹⁸F-FDG standardized uptake value (SUV) compared to rats administered saline, indicating increased glucose retention and utilization in myocardial tissue. Imaging with the ¹⁸F-FTHA PET tracer shows an initial ˜2-fold increase after CV-8972 administration (from ¹⁸F-FTHA circulating in the blood compartment) followed by rapid tissue absorption and a washout of ¹⁸F-FTHA from rat heart tissue over the 120-minute image acquisition. Furthermore, a static analysis at 30 minutes shows a 12-fold increase in the ¹⁸F-FDG SUV in rat heart tissue compared with a minimal change in ¹⁸F-FDG SUV in skeletal muscle. These data suggest that CV-8972 triggers a switch in cardiomyocytes from energy generation based on fatty acid oxidation to glucose dependent metabolism.

In a toxicokinetic study with subcutaneous injection of CV-8972 at 60 mg/kg to male rats, the exposures (AUC 0-8 hr) of CV-8814 and TMZ were 72.5 and 2.7 nmol·hr/ml, respectively. Therefore, CV-8814 plasma AUC represented 96.4% of the combined exposure for CV-8814 and TMZ, and the pharmacologic effect predominantly reflected that of CV-8814.

### Example 2

Studies were conducted to evaluate the effect of CV-8814, a pFOX inhibitor, on protecting heart tissue from ischemia/reperfusion (I/R) injury. Similar studies were performed with trimetazidine (TMZ) as a representative pFOX reference compound. Studies were performed with TMZ alone and TMZ in combination with the metabolic enhancers (e.g., nicotinic acid) for a comparison of single agent and combination agent pharmacologic potencies.

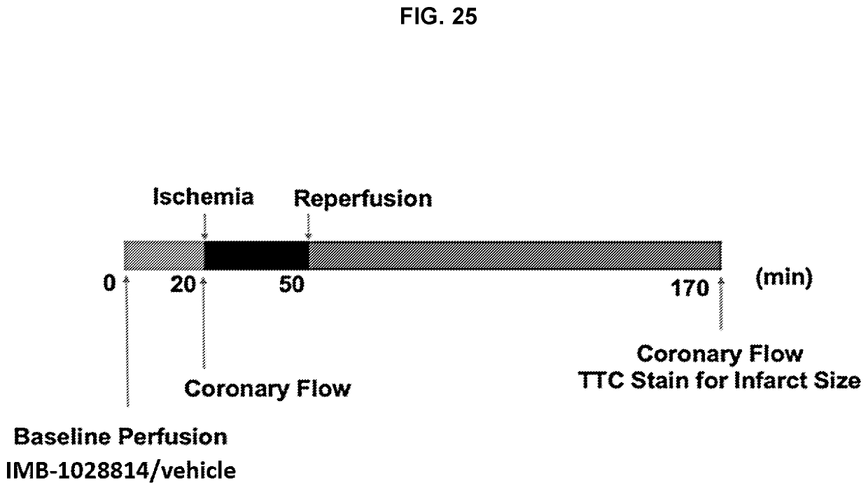

FIG. 25 is a schematic of the Langendorff Ischemia-Reperfusion protocol used to test the ability of various compounds to protect the heart from ischemic injury. Test compounds were dissolved at 20 μM in Krebs-Henseleit buffer and perfused through the tissue of mouse hearts at a constant pressure starting at time 0. For each treatment group, 10-18 hearts were tested. A Mikro-tip catheter was inserted into left ventricles to measure cardiac functions, including heart rate, coronary flow, left ventricular systolic and end-diastolic pressure, at the end of the baseline perfusion at 20 minutes. Heart were reperfused at 50 minutes, and cardiac functions were measured again at 170 minutes. At the end of perfusion, hearts were cross-sectioned into five slices, and infarct size was measured by computerized planimetry of triphenyltetrazolium stained heart tissue sections.

In an initial study, the single agent activity of CV-8814 was tested.

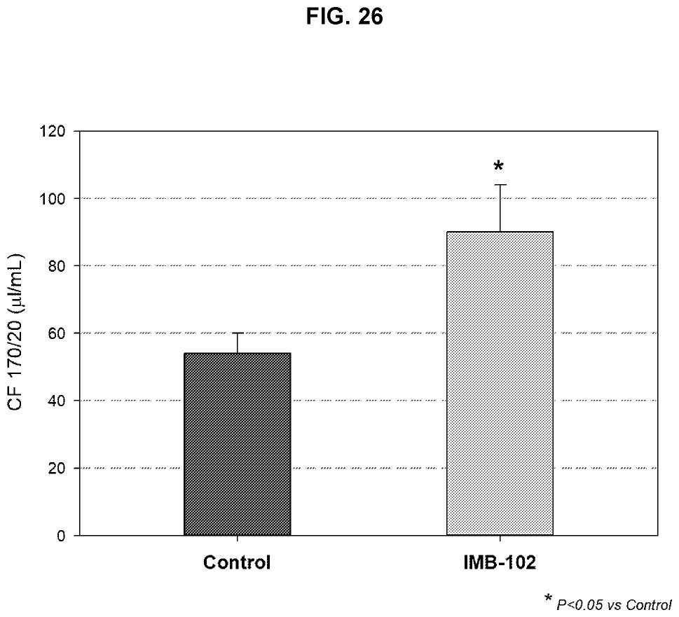

FIG. 26 is a graph of coronary flow during ischemia-reperfusion injury in explanted mouse hearts treated with either saline (Control, blue bar) or 20 μM CV-8814 (IMB-102, orange bar). * p<0.05 vs. Control.

CV-8814 perfusion significantly increased coronary flow measured at the end of the reperfusion period (CV-8814: 90±14 μl/mL vs control: 54±6 μl/mL, p<0.05) suggesting that CV-8814 protected coronary vessels from ischemic injury.

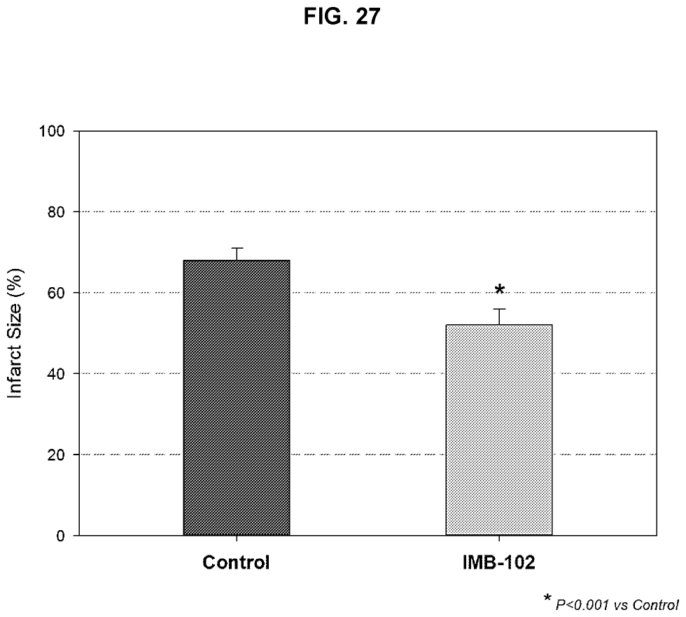

FIG. 27 is a graph of infarct size following ischemia-reperfusion injury in explanted mouse hearts treated with either saline (Control, blue bar) or 20 μM CV-8814 (IMB-102, orange bar). * p<0.001 vs. Control.

CV-8814 perfusion preserved cardiac function as measured by left ventricular developed pressure (LVDP; 48±8 mmHg at 20 min vs. 63±5 mm Hg at 170 min, p<0.05) compared to the significant decrease in LVDP observed in control hearts (33±3 mmHg at 20 min vs. 56±3 mm Hg at 170 min, p<0.001). CV-8814 perfusion also protected myocardial tissue from ischemia associated cell death as measured by a reduction in infarct size (CV-8814: 52±4% vs. control: 68±3%, p<0.001).

These results indicate that CV-8814 perfusion significantly reduced the myocardial tissue area at risk of ischemic injury while sustaining coronary flow and cardiac function as measured in the Langendorff I/R model.

Comparative data with reference compound TMZ alone were generated in a separate study.

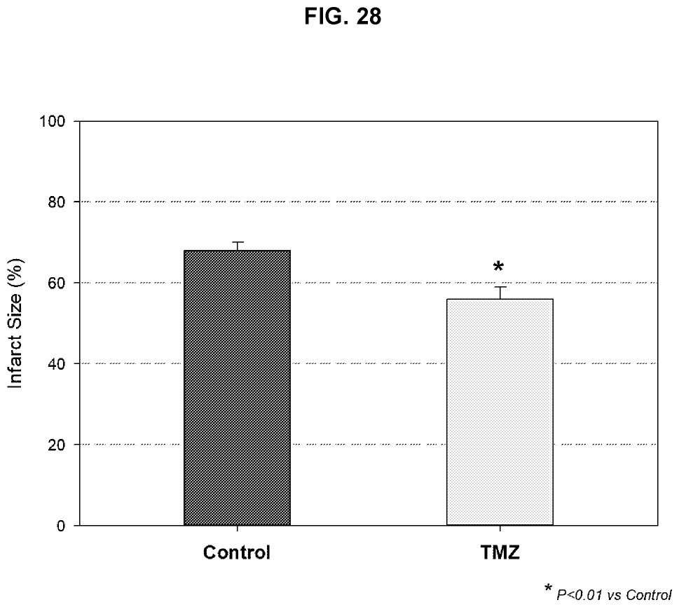

FIG. 28 is a graph of infarct size following ischemia-reperfusion injury in explanted mouse hearts treated with either saline (Control, blue bar) or 20 μM trimetazidine (TMZ, aqua bar). * p<0.01 vs. Control.

The results show that TMZ alone had comparable effects in preserving coronary flow (TMZ vs. control: 90±1.0 mL/min vs. 60±1.0 mL/min, p<0.05) and LVDP (TMZ vs. control: 49±5 vs. 56±6 mm Hg, p>0.05). TMZ also reduced myocardial infarct size, although only by ˜9%, compared with ˜16% for CV-8814.

The concept of combining a pFOX inhibitor with metabolic enhancers (e.g. nicotinamide, nicotinic acid, and succinate) was evaluated in combination Langendorff I/R studies. The combinations included TMZ/nicotinamide/succinate (all at a concentration of 20 μM; coded TNS) and TMZ/nicotinic acid/succinate (all at a concentration of 20 μM; coded TNC).

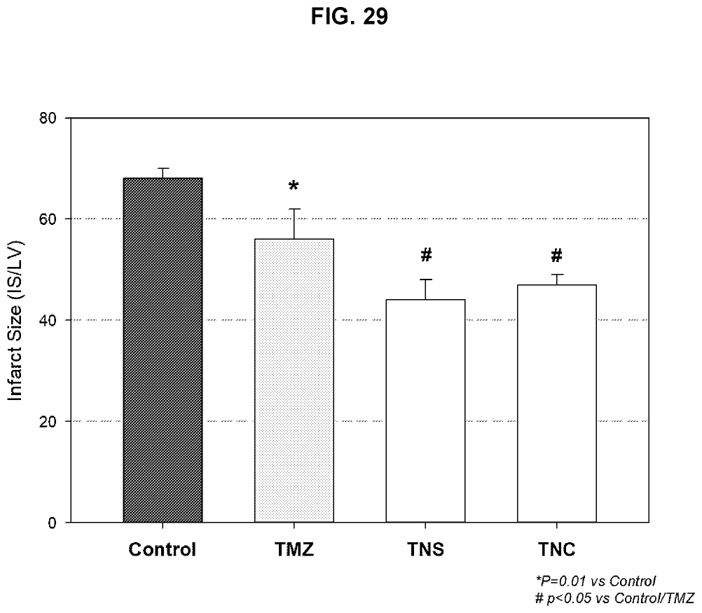

FIG. 29 is a graph of infarct size following ischemia-reperfusion injury in explanted mouse hearts treated with saline (Control, blue bar), 20 μM trimetazidine (TMZ, aqua bar), 20 μM each of trimetazidine+nicotinamide+succinate (TNS, white bar), or 20 μM each of trimetazidine+nicotinic acid+succinate (TNC, white bar). * P=0.01 vs. Control, #p<0.05 vs. Control/TMZ.

Perfusion with 20 μM TMZ/nicotinamide/succinate (TNS) provided the most significant increase in coronary flow (TNS vs. control: 62±9 μL/mL vs. 36±3 μl/mL, p<0.05) while TMZ/nicotinic acid/succinate (TNC: 54+9 μL/mL) was more effective than TMZ alone (40+6 μL/mL). The TNS triple combination and TMZ alone both sustained cardiac function measured by LVDP (TNS: T170 vs. T20=48±8 vs. 66±5 mm Hg, p>0.05; TMZ: T170 vs. T20=49±5 vs. 56±6 mm Hg, p>0.05). The TNS and TNC triple combination perfusions both protected myocardial tissue from ischemia associated cell death as measured by a reduction in infarct size (TNS vs. control: 44±4% vs. 68±2%, p<0.001; TNC vs. control: 47±2% vs. 68±2%, p<0.001). The TNS and TNC combinations were both more effective than TMZ alone (56±3% infarcted tissue area).

### Example 3

Studies were conducted to evaluate and compare the efficacy of the reference compound TMZ with CV-8814 and CV-1018972 on ventricular remodeling, preservation of heart function and prevention of fibrosis in a transverse aortic constriction (TAC) model of heart failure in mice.

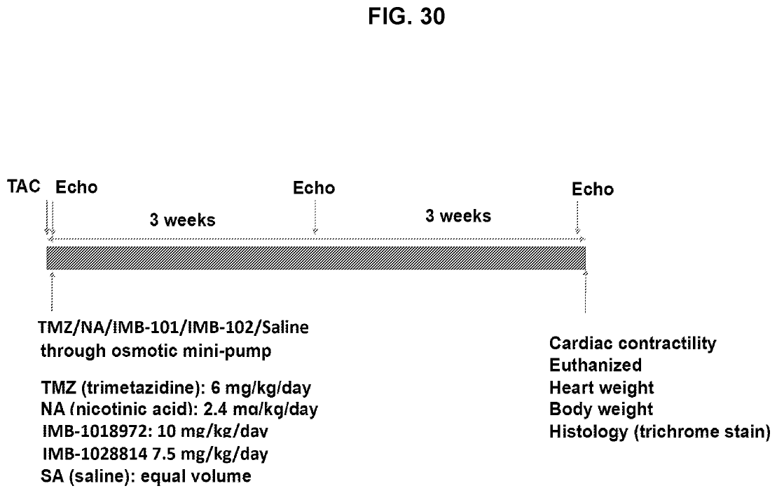

FIG. 30 is a schematic of the transverse aortic constriction (TAC) protocol used to test the ability of various compounds to protect the heart against heart failure. The aortic arc was exposed via midline incision in the chest cavity of anesthetized mice. A 27-gauge needle was tied against the transverse aorta and then promptly removed to create a ligature constriction. Treatments were administered through a subcutaneous osmotic minipump at the concentrations indicated in the figure. In vivo cardiac function was assessed by transthoracic echocardiography performed at 24-hour, 3-week, and 6-week time-points after TAC. Ventricular remodeling was determined by evaluation of heart weights (HW) and heart weight to body weight ratios (HW/BW). At study termination, hearts were excised, fixed, sectioned at 6 μm and stained with Masson's trichome. The area of fibrosis in myocardial tissue was measured by computerized planimetry on five random fields per heart. A total of 65 to 75 fields were analyzed per treatment group and differences were analyzed by Student's t-test.

Three separate studies were performed to evaluate the efficacy of single agents and combinations with metabolic enhancers in the TAC model of heart failure. In the first study, mice were treated with either trimetazidine alone or trimetazidine+nicotinamide+succinate. In the second study, mice were treated with CV-8814 alone or trimetazidine+nicotinic acid+succinate. In the third study, mice were treated with nicotinic acid, CV-8814, or CV-8972. Overall, the activities of trimetazidine, CV-8814, and CV-8972 on most study endpoints were in good agreement across the three studies. In the third study, the three compounds were analyzed at equivalent mg/kg/day doses on a formula weight basis: 6 mg/kg/d for trimetazidine, 7.5 mg/kg/day for CV-8814; and 10 mg/kg/day for CV-8972. Therefore, the results from the third study are summarized here.

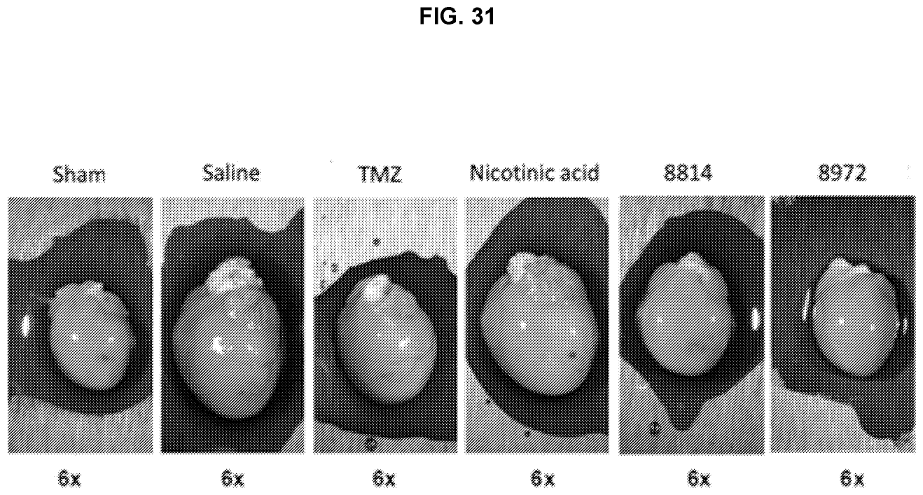

FIG. 31 shows images of hearts from mice following TAC-induced heart failure. Left panel shows the heart from a mouse that was given a sham procedure in which TAC was not performed. The remaining panels shows hearts from mice treated with saline (Saline), trimetazidine (TMZ), nicotinic acid (Nicotinic acid), CV-8814 (8814), or CV-8972 (8972).

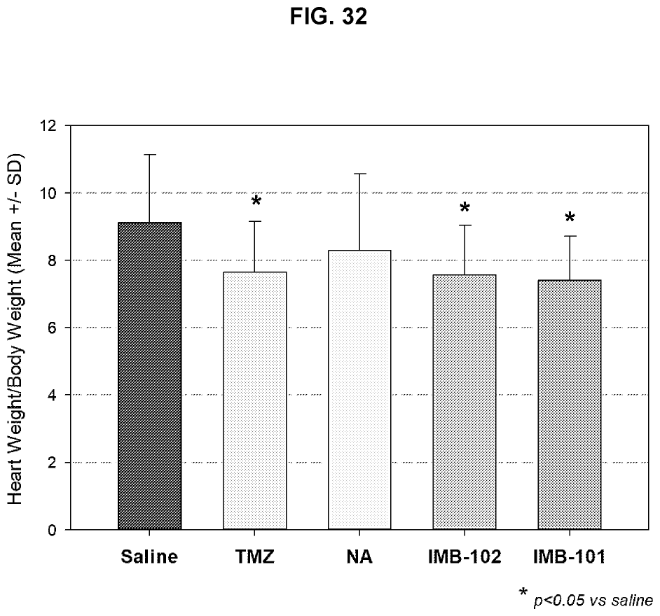

FIG. 32 is a graph of heart weight to body weight following TAC-induced heart failure in mice treated with saline (Saline, blue bar), trimetazidine (TMZ, aqua bar), nicotinic acid (NA, light green bar), CV-8814 (IMB-102, light orange bar), or CV-8972 (IMB-101, dark orange bar). Values of heart weight to body weight are expressed as mg/g. * p<0.05 vs. saline treatment.

Administration of CV-8972 had a significant effect on ventricular remodeling and cardiac functions in the TAC model of heart failure. Compared to the control group, CV-8972 prevented cardiac hypertrophy as measured by a reduction in heart weight (CV-8972 vs. control: 225±11 mg vs. 270±14 mg; p<0.05), heart weight to body weight ratio (CV-8972 vs. control: 7.4±0.3 mg/g vs. 9.1±0.5 mg/g; p<0.05) and a reduction in left ventricular mass (CV-8972 vs. control: 156±10 mg vs. 195±12 mg; p<0.05). The effect of TMZ or CV-8814 treatments were similar to CV-8972.

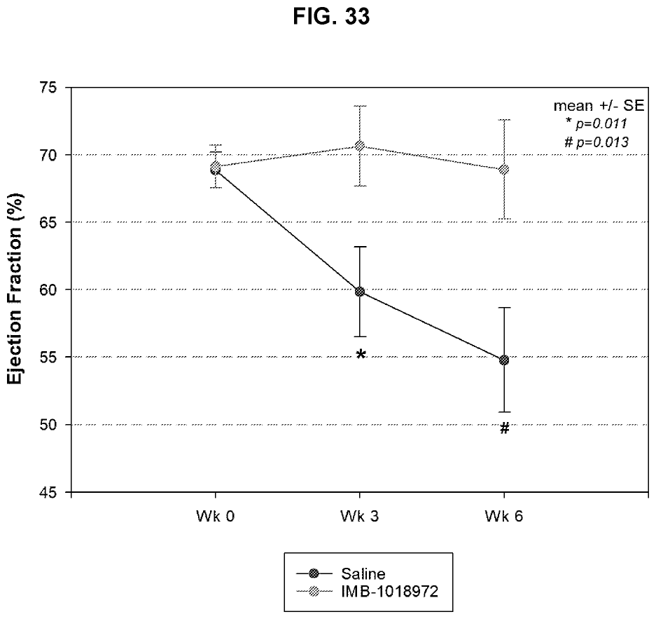

FIG. 33 is a graph of cardiac ejection fraction at various time points following TAC-induced heart failure in mice treated with either saline (Saline, blue circles) or CV-8972 (CV-8972, orange circles). * p=0.011, #p=0.013.

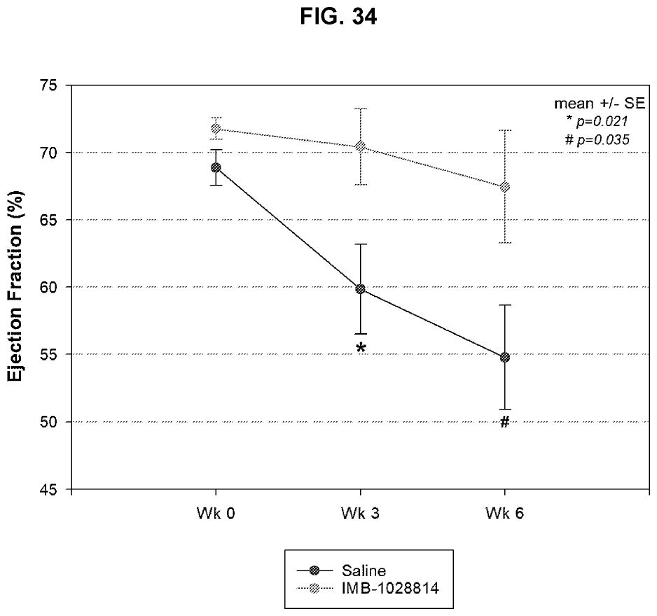

FIG. 34 is a graph of cardiac ejection fraction at various time points following TAC-induced heart failure in mice treated with either saline (Saline, blue circles) or CV-8814 (CV-8814, orange circles). * p=0.021, #p=0.035.

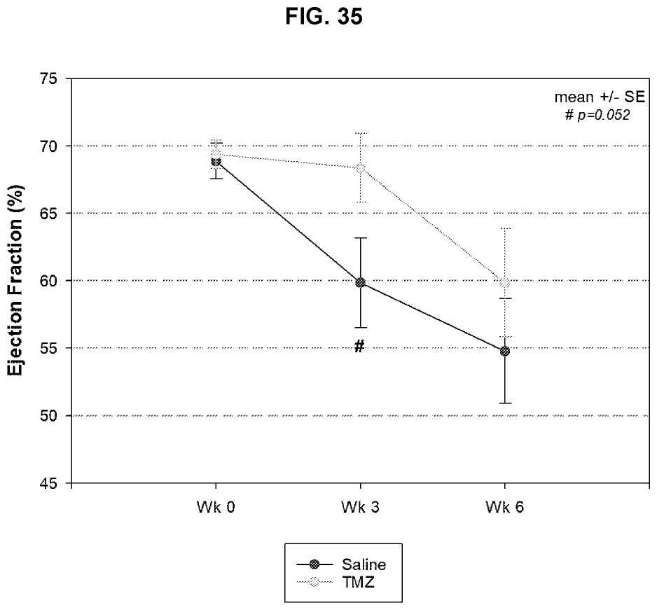

FIG. 35 is a graph of cardiac ejection fraction at various time points following TAC-induced heart failure in mice treated with either saline (Saline, blue circles) or trimetazidine (TMZ, aqua circles). #p=0.052.

CV-8972 administration also preserved cardiac function in mice over the six-week study period as measured by increased left ventricular fractional shortening (FS) measured at Week 3 (CV-8972 vs. control: 47%±3% vs. 37%±3%; p<0.05). FS continued to decline in control TAC animals to Week 6 (34%±3%) while the effect of both CV-8972 (46%±3%; p<0.05) and CV-8814 (44%±3%; p<0.05) on FS was sustained. In contrast, TMZ did not have a significant effect on FS. Treatment with both CV-8972 and CV-8814 also preserved left ventricular ejection fraction (EF) throughout the six-week study while neither TMZ nor nicotinic acid had a beneficial effect.

The treatment effect of CV-8972 and CV-8814 on diastolic function during TAC-induced heart failure was assessed by measuring isovolumic relaxation time (IVRT). Both CV-8972 and CV-8814 prevented the prolongation of IVRT at the three-week assessment (CV-8972 vs. control: 32±1 ms vs. 36±1 ms; p<0.05; CV-8814 vs. control: 33±1 ms vs. 36±1 ms; p<0.05). The effect of CV-8814 was sustained to Week 6 (CV-8814 vs. control: 28±2 ms vs. 35±1 ms, p<0.0) while CV-8972 was slightly less effective (CV-8972 vs. control: 31±2 ms vs. 35±1 ms, p=0.06). In contrast, TMZ treatment did not preserve diastolic function during TAC-induced heart failure.

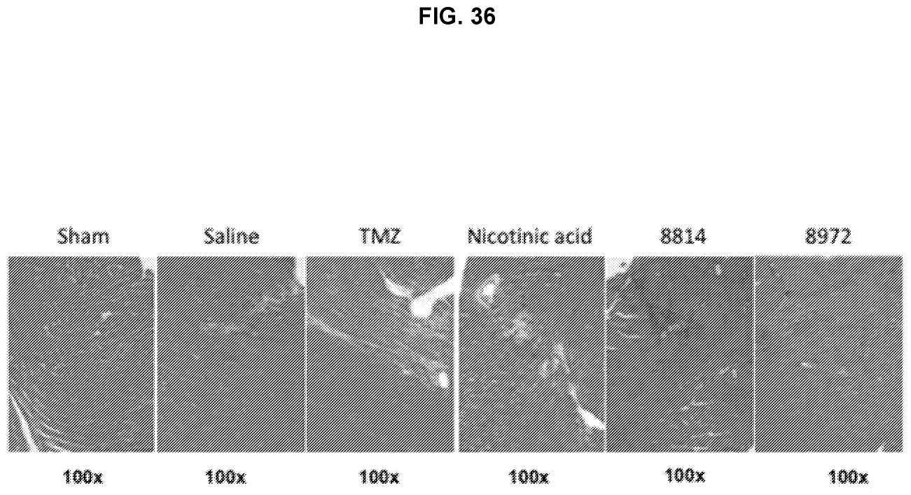

FIG. 36 shows microscopic images of heart tissue from mice following TAC-induced heart failure. Left panel shows heart tissue from a mouse that was given a sham procedure in which TAC was not performed. The remaining panels show hearts from mice treated with saline (Saline), trimetazidine (TMZ), nicotinic acid (Nicotinic acid), CV-8814 (8814), or CV-8972 (8972).

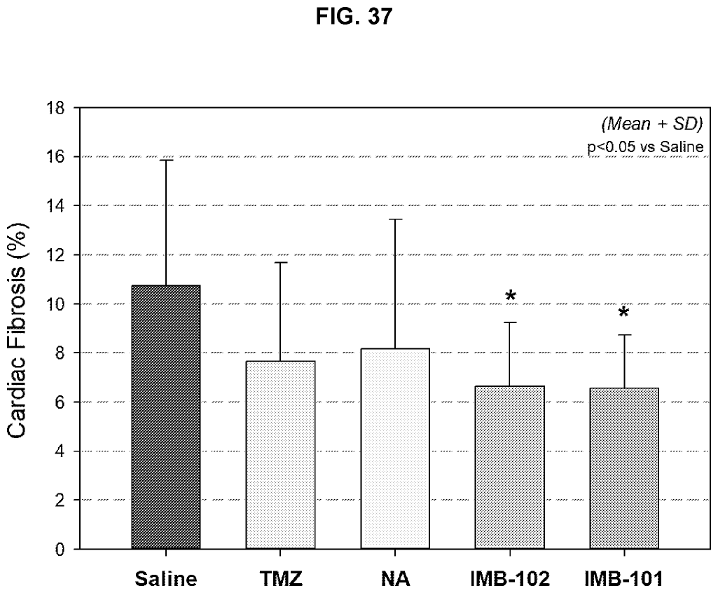

FIG. 37 is graph of cardiac fibrosis following TAC-induced heart failure in mice treated with saline (Saline, blue bar), trimetazidine (TMZ, aqua bar), nicotinic acid (NA, light green bar), CV-8814 (IMB-102, light orange bar), or CV-8972 (IMB-101, dark orange bar). Values represent percentage of fibrotic heart tissue. *p<0.05 vs. saline treatment.

Finally, CV-8972 administration significantly reduced fibrosis of myocardial tissue after six weeks of TAC-induced heart failure (CV-8972 vs. control: 6.6±0.6 vs. 10.7±1%; p<0.01). The effect of CV-8814 was similar (6.6±0.6%; p<0.01) and both CV-8972 and CV-8814 were more effective than TMZ (7.6±1% vs. 11±1%; p=0.08) in preventing myocardial fibrosis. Nicotinic acid alone had a modest effect on fibrosis (8.2±1%).

Based on a pharmacokinetic study with a 10 mg/kg intravenous administration of CV-8972, CV-8972 is rapidly converted to CV-8814, with low, but measurable levels of TMZ. Based on the plasma exposure (AUC 0-8 hr) CV-8814 represented 93.9% of the combined AUC for CV-8814 and TMZ. Therefore, in vivo pharmacologic effects in mice with parenteral dosing of CV-8972 predominantly reflect activity of CV-8814 in this species

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A combination therapy comprising:
a compound represented by formula (X): 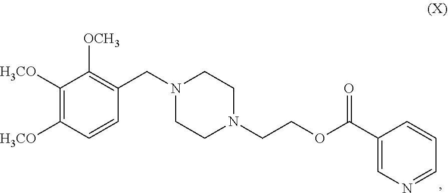 [SMILES: COc1ccc(CN2CCN(CCOC(=O)c3cccnc3)CC2)c(OC)c1OC]
or a pharmaceutically acceptable salt thereof, and
a compound represented by formula (IX): 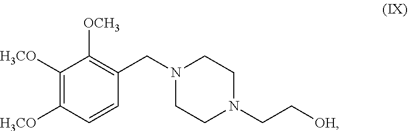 [SMILES: COc1ccc(CN2CCN(CCO)CC2)c(OC)c1OC]
or a pharmaceutically acceptable salt thereof.

2. The combination therapy of claim 1, wherein the combination therapy comprises the compound of formula (X) or salt thereof and the compound of formula (IX) or salt thereof in a defined mass ratio.

3. The combination therapy of claim 2, wherein the mass ratio of the compound of formula (X) or salt thereof to the compound of formula (IX) or salt thereof is from about 10:1 to about 1:100.

4. The combination therapy of claim 3, wherein the mass ratio is from about 1:1 to about 1:10.

5. The combination therapy of claim 1, wherein the combination therapy comprises:
a daily dosage of the compound of formula (X) or salt thereof of from about 50 mg to about 1000 mg,
a daily dosage of the compound of formula (IX) or salt thereof of from about 50 mg to about 1000 mg.

6. The combination therapy of claim 5, wherein the daily dosage of the compound of formula (X) or salt thereof is from about 100 mg to about 500 mg.

7. The combination therapy of claim 5, wherein the daily dosage of the compound of formula (IX) or salt thereof is from about 100 mg to about 500 mg.

8. The combination therapy of claim 1, wherein the combination therapy comprises the compound of formula (X) or salt thereof and the compound of formula (IX) or salt thereof in separate formulations.

9. The combination therapy of claim 1, wherein the combination therapy comprises the compound of formula (X) or salt thereof and the compound of formula (IX) or salt thereof in a single formulation.

10. The combination therapy of claim 1, wherein the combination therapy is suitable for treatment of a disease, disorder, or condition selected from the group consisting of acute coronary syndrome, acute heart failure, advanced heart failure, aneurysm, angina, anthracycline-induced cardiotoxicity, atherosclerosis, cardiac allograft vasculopathy, cardiac steatosis, cardiac transplant vasculopathy, cardiomyopathy, cerebral vascular disease, chronic coronary syndrome, chronic heart failure, congenital heart disease, contrast nephropathy, coronary artery disease (CAD), coronary heart disease, diabetic cardiomyopathy, dilated cardiomyopathy (DCM, including idiopathic, heart attack, heart disease, heart failure with mildly reduced ejection fraction (HFmrEF), heart failure with preserved ejection fraction (HFpEF), heart failure with reduced ejection fraction (HFrEF), heart failure, hibernating myocardium, high blood pressure (hypertension), hypertrophic cardiomyopathy (HCM, including non-obstructive or obstructive), intermittent claudication, ischemia with non-obstructive coronary arteries (INOCA), ischemia-reperfusion injury, ischemic cardiomyopathy, ischemic heart disease, microvascular angina, myocardial dysfunction induced by anti-cancer drugs, myocardial infarction with non-obstructive coronary arteries (MINOCA), myocarditis, non-familial and familial/genetic), non-ischemic cardiomyopathy, pericardial disease, peripartum cardiomyopathy, peripheral arterial disease, peripheral vascular disease, pulmonary arterial hypertension, pulmonary hypertension, refractory angina, restrictive cardiomyopathy, rheumatic heart disease, right heart failure, right ventricular failure, stable angina, stroke, stunned myocardium, tachycardiomyopathy, Takotsubo cardiomyopathy, transient ischemic attack, unstable angina, or valvular heart disease.

11. A method of treating a disease, disorder, condition in a subject, the method comprising providing to a subject having a disease, disorder, or condition:
a compound represented by formula (X): 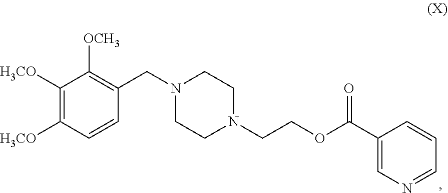 [SMILES: COc1ccc(CN2CCN(CCOC(=O)c3cccnc3)CC2)c(OC)c1OC]
or a pharmaceutically acceptable salt thereof, and
a compound represented by formula (IX): 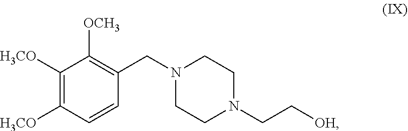 [SMILES: COc1ccc(CN2CCN(CCO)CC2)c(OC)c1OC]
or a pharmaceutically acceptable salt thereof.

12. The method of claim 11, wherein the compound of formula (X) or salt thereof and the compound of formula (IX) or salt thereof are provided in a defined mass ratio.

13. The method of claim 12, wherein the mass ratio of the compound of formula (X) or salt thereof to the compound of formula (IX) or salt thereof is from about 10:1 to about 1:100.

14. The method of claim 13, wherein the mass ratio is from about 1:1 to about 1:10.

15. The method of claim 11, wherein:
the compound of formula (X) or salt thereof is provided in a daily dosage of from about 50 mg to about 1000 mg, and
the compound of formula (IX) or salt thereof is provided in a daily dosage of from about 50 mg to about 1000 mg.

16. The method of claim 15, wherein the compound of formula (X) or salt thereof is provided in a daily dosage of from about 100 mg to about 500 mg.

17. The method of claim 15, wherein the compound of formula (IX) or salt thereof is provided in a daily dosage of from about 100 mg to about 500 mg.

18. The method of claim 11, wherein the compound of formula (X) or salt thereof and the compound of formula (IX) or salt thereof are provided in separate formulations.

19. The method of claim 11, wherein the compound of formula (X) or salt thereof and the compound of formula (IX) or salt thereof are provided in a single formulation.

20. The method of claim 11, wherein the disease, disorder, or condition is selected from the group consisting of acute coronary syndrome, acute heart failure, advanced heart failure, aneurysm, angina, anthracycline-induced cardiotoxicity, atherosclerosis, cardiac allograft vasculopathy, cardiac steatosis, cardiac transplant vasculopathy, cardiomyopathy, cerebral vascular disease, chronic coronary syndrome, chronic heart failure, congenital heart disease, contrast nephropathy, coronary artery disease (CAD), coronary heart disease, diabetic cardiomyopathy, dilated cardiomyopathy (DCM, including idiopathic, heart attack, heart disease, heart failure with mildly reduced ejection fraction (HFmrEF), heart failure with preserved ejection fraction (HFpEF), heart failure with reduced ejection fraction (HFrEF), heart failure, hibernating myocardium, high blood pressure (hypertension), hypertrophic cardiomyopathy (HCM, including non-obstructive or obstructive), intermittent claudication, ischemia with non-obstructive coronary arteries (INOCA), ischemia-reperfusion injury, ischemic cardiomyopathy, ischemic heart disease, microvascular angina, myocardial dysfunction induced by anti-cancer drugs, myocardial infarction with non-obstructive coronary arteries (MINOCA), myocarditis, non-familial and familial/genetic), non-ischemic cardiomyopathy, pericardial disease, peripartum cardiomyopathy, peripheral arterial disease, peripheral vascular disease, pulmonary arterial hypertension, pulmonary hypertension, refractory angina, restrictive cardiomyopathy, rheumatic heart disease, right heart failure, right ventricular failure, stable angina, stroke, stunned myocardium, tachycardiomyopathy, Takotsubo cardiomyopathy, transient ischemic attack, unstable angina, or valvular heart disease.

21. A pharmaceutical formulation comprising:
a compound represented by formula (X): 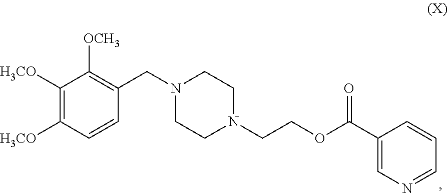 [SMILES: COc1ccc(CN2CCN(CCOC(=O)c3cccnc3)CC2)c(OC)c1OC]
or a pharmaceutically acceptable salt thereof, and
a compound represented by formula (IX): 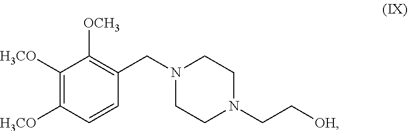 [SMILES: COc1ccc(CN2CCN(CCO)CC2)c(OC)c1OC]
or a pharmaceutically acceptable salt thereof.

22-30. (canceled)
